(19) United States
(12) Patent Application Publication
(10) Pub. No.: US 2025/0131981 A1
(43) Pub. Date: Apr. 24, 2025

(54) METHODS AND SYSTEMS FOR DETERMINING MICROSATELLITE INSTABILITY

(71) Applicant: Foundation Medicine, Inc., Cambridge, MA (US)

(72) Inventors: Kyle GOWEN, Arlington, MA (US); Jason HUGHES, Cambridge, MA (US); Meijuan LI, Clarksville, MD (US); Lei YANG, Boston, MA (US); Jeffrey LEIBOWITZ, Sudbury, MA (US); Pei MA, Wilmington, MA (US)

(73) Assignee: Foundation Medicine, Inc., Cambridge, MA (US)

(21) Appl. No.: 18/579,296

(22) PCT Filed: Jul. 14, 2021
(86) PCT No.: PCT/US2021/041643

§ 371 Date: Jan. 12, 2024

(51) Int. Cl. G16B 20/20; C12Q 1/6869; G16H 20/10; G16H 50/20; G16H 50/30

(52) U.S. Cl. CPC G16B 20/20; C12Q 1/6869; G16H 20/10; G16H 50/20; G16H 50/30

(57) ABSTRACT

Methods for detecting a deficient DNA repair mechanism and/or evaluating microsatellite instability in a sample from a subject are described. The disclosed methods comprise analyzing nucleic acid sequence data for a plurality of microsatellite loci to calculate a microsatellite instability (MSI) score for the sample from a ratio of the number of microsatellite loci that exhibit variant alleles to the total number of microsatellite loci that meet, e.g., a minimum sequencing coverage requirement. The MSI score is compared a first threshold and, m some cases, to a second threshold to assign a status of high microsatellite instability′(MSI-H), microsatellite stable (MSS), or equivocal microsatellite instability (MSI-E) for the sample.

### BACKGROUND

Microsatellites regions of the genome are short DNA motifs (typically about 1-6 base pairs in length, but sometimes comprising motifs of 15 or more base pairs in length) that repeat in tandem (typically about 5-50×). There are over 600,000 unique microsatellite loci in the human genome. While many are located in non-coding regions of the genome, they can also be located in regulatory regions and coding regions.

Microsatellite instability (MSI) is a condition of genetic predisposition to mutation in the microsatellite regions that results from, e.g., slipped strand mismatch during DNA replication and an impaired DNA mismatch repair (MMR) mechanism. Microsatellite instability is conventionally evaluated by means of PCR amplification and amplicon size determination using primers that target the DNA sequences flanking a panel of microsatellite loci. However, the difficulty and cost of developing primers that function properly in a multiplexed reaction places a practical limit on the number of microsatellite loci that may be interrogated simultaneously, and limits the robustness of such assays. PCR-based methods for determining MSI status also often require a matched normal tissue sample for every specimen assayed because rare germline polymorphisms may be misinterpreted as positive microsatellite loci. Existing sequencing-based approaches to determining MSI status are sensitive to germline variation and susceptible to noisy or artifactual signals derived from sequencing reads for a relatively small set of genomic loci. Therefore, there is a need for improved methods for evaluating microsatellite instability in patient samples.

### BRIEF SUMMARY OF THE INVENTION

Disclosed herein are methods, systems, and non-transitory storage media for evaluating microsatellite instability status of a sample based on nucleic acid sequencing data and a per-locus analysis of variant allele frequencies (i.e., the frequencies for variant alleles of altered length) at a plurality of microsatellite loci. Several filters are used to exclude candidate alleles that are likely to be the result of noise, sequencing errors, or that are germline alleles. The input microsatellite allele sequences are individually categorized as stable or unstable, and an MSI score for the sample is calculated by dividing the number of unstable loci (i.e., loci exhibiting at least one unstable allele) by the total number of loci evaluated for allelic stability (e.g., the total number of loci that met a specified minimum sequencing coverage requirement) to determine the fraction of microsatellite loci that are unstable. A threshold is then applied to the MSI score for classification of the sample as microsatellite instability—high (MSI-H), microsatellite instability—equivocal (MSI-E), or microsatellite stable (MSS).

The disclosed methods exhibit increased sensitivity and accuracy compared to PCR-based methods or existing sequencing-based methods for determining MSI status due to the large number of microsatellite loci that may be interrogated and the enforcement of minimum sequencing coverage requirements and minimum microsatellite allele frequency requirements. The methods exhibit reduced sensitivity to germline variation, improved robustness against confounding effects (e.g., processing-related issues or other effects that lead to increased noise or detection of artifactual signal), an improved limit of detection (LoD), and eliminate the need for a matched normal sample while taking advantage of automated or semi-automated sequencing analysis processes to reduce analysis cost and improve inter-laboratory variability.

In some instances, the disclosed methods for determining microsatellite instability status may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for determining microsatellite instability as part of a genomic profiling process can improve the validity of, e.g., disease detection calls, made on the basis of the genomic profiling by, for example, independently confirming the presence of an impaired DNA mismatch repair (MMR) mechanism in a given patient sample. In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

Disclosed herein are methods for detecting a deficient DNA mismatch repair mechanism in a sample from a subject, the methods comprising: providing a plurality of nucleic acid molecules obtained from a sample from the subject; ligating one or more adapters onto one or more nucleic acid molecules from the plurality of nucleic acid molecules; amplifying nucleic acid molecules from the plurality of nucleic acid molecules, capturing nucleic acid molecules from the amplified nucleic acid molecules; sequencing, by a sequencer, the captured nucleic acid molecules to obtain a plurality of sequence reads corresponding to a plurality of microsatellite loci; identifying, by one or more processors, a set of microsatellite loci from the plurality of microsatellite loci based on a coverage requirement; applying, by the one or more processors, a set of sequence-based exclusion criteria to the set of microsatellite loci to identify a subset of the set of microsatellite loci; determining, by the one or more processors, a microsatellite instability (MSI) score for the sample based on a number of microsatellite loci in the set and a number of microsatellite loci in the subset; comparing, by the one or more processors, the MSI score to a threshold; and calling an MSI status of high microsatellite instability for the sample if the MSI score is greater than or equal to the threshold, wherein the MSI status of high microsatellite instability is indicative of a deficient DNA mismatch repair mechanism in a tissue of the subject.

In some embodiments, the subject is a cancer patient. In some embodiments, the sample comprises a tissue sample, a biopsy sample, a liquid biopsy sample, a hematological sample (e.g., bone marrow), or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the plurality of microsatellite loci comprises at least 500 loci. In some embodiments, the plurality of microsatellite loci comprises at least 1,000 loci. In some embodiments, the plurality of microsatellite loci comprises at least 1,500 loci. In some embodiments, the plurality of microsatellite loci comprises between 100 and 3.000 loci, between 200 and 2,800 loci, between 300 and 2,600 loci, between 400 and 2,400 loci, between 500 and 2,200 loci, between 600 and 2.000 loci, between 700 and 1.800 loci, between 800 and 1.600 loci, between 900 and 1,400 loci, between 1,000 and 1,200 loci, between 400 and 1,000 loci, between 400 and 1,200 loci, between 400 and 1,400 loci, between 400 and 1,600 loci, between 400 and 1,800 loci, between 400 and 2,000 loci, between 400 and 2,200 loci, between 400 and 2,400 loci, between 400 and 2,600 loci, between 400 and 2,800 loci, between 400, and 3,000 loci, between 600 and 1,000 loci, between 600 and 1,200 loci, between 600 and 1,400 loci, between 600 and 1,600 loci, between 600 and 1.800 loci, between 600 and 2,000 loci, between 600 and 2,200 loci, between 600 and 2,400 loci, between 600 and 2,600 loci, between 600 and 2,800 loci, between 600, and 3,000 loci, between 800 and 1,000 loci, between 800 and 1,200 loci, between 800 and 1,400 loci, between 800 and 1,600 loci, between 800 and 1,800 loci, between 800 and 2,000 loci, between 800 and 2,200 loci, between 800 and 2,400 loci, between 800 and 2,600 loci, between 800 and 2,800 loci, between 800, and 3,000 loci, between 1,000 and 1,200 loci, between 1.000 and 1,400 loci, between 1,000 and 1,600 loci, between 1,000 and 1,800 loci, between 1,000 and 2,000 loci, between 1,000 and 2,200 loci, between 1,000 and 2,400 loci, between 1,000 and 2,600 loci, between 1,000 and 2,800 loci, between 1,000, and 3.000 loci, between 1,200 and 1,400 loci, between 1,200 and 1,600 loci, between 1,200 and 1,800 loci, between 1,200 and 2,000 loci, between 1,200 and 2,200 loci, between 1,200 and 2,400 loci, between 1,200 and 2,600 loci, between 1,200 and 2,800 loci, between 1,200, and 3,000 loci, between 1,400 and 1,600 loci, between 1,400 and 1.800 loci, between 1.400 and 2.000 loci, between 1,400 and 2,200 loci, between 1,400 and 2,400 loci, between 1,400 and 2,600 loci, between 1,400 and 2,800 loci, between 1,400, and 3,000 loci, between 1,600 and 1,800 loci, between 1,600 and 2,000 loci, between 1,600 and 2,200 loci, between 1,600 and 2,400 loci, between 1,600 and 2,600 loci, between 1,600 and 2,800 loci, between 1,600, and 3,000 loci, between 1,800 and 2,000 loci, between 1,800 and 2,200 loci, between 1,800 and 2,400 loci, between 1,800 and 2,600 loci, between 1,800 and 2,800 loci, between 1,800, and 3,000 loci, between 2,000 and 2,200 loci, between 2,000 and 2.400 loci, between 2.000 and 2.600 loci, between 2,000 and 2,800 loci, between 2,000 and 3,000 loci, between 2,200 and 2,400 loci, between 2,200 and 2,600 loci, between 2,200 and 2,800 loci, between 2,200, and 3,000 loci, between 2,400 and 2,600 loci, between 2,400 and 2,800 loci, between 2,400, and 3,000 loci, between 2,600 and 2,800 loci, between 2,600, and 3,000 loci, or between 2,800 and 3,000 loci. In some embodiments, the microsatellite loci comprise alleles having mononucleotide, dinucleotide, or trinucleotide repeat sequences. In some embodiments, the one or more adapters comprise amplification primers, sequencing adapters, sample index sequences, or any combination thereof. In some embodiments, the captured nucleic acid molecules are captured from the amplified nucleic acid molecules by hybridization to one or more bait molecules and the one or more bait molecules comprise one or more nucleic acid molecules, each comprising a region that is complementary to a region of a captured nucleic acid molecule. In some embodiments, amplifying nucleic acid molecules comprises performing a polymerase chain reaction (PCR) or isothermal amplification technique. In some embodiments, the sequencing comprises use of a next generation sequencing (NGS) technique. In some embodiments, the sequencer comprises a next generation sequencer. In some embodiments, the coverage requirement is at least 75×, 100×, 150×, 150×, 200×, or 250×. In some embodiments, applying the set of sequence-based exclusion criteria comprises excluding, from the set of microsatellite loci, any microsatellite locus that comprises an allele having an allele frequency below an allele frequency requirement. In some embodiments, the MSI score is calculated as a ratio of the number of microsatellite loci in the subset to the number of microsatellite loci in the set. In some embodiments, the threshold is a first threshold, and the method further comprises: comparing the MSI score to a second threshold if the MSI score is less than the first threshold; and calling an MSI status of microsatellite stable for the sample if the MSI score is less than or equal to the second threshold; or calling an MSI status of equivocal microsatellite instability for the sample if the MSI score is greater than the second threshold. In some embodiments, the method further comprises: generating a report of the MSI status, displaying the report of the MSI status on a display device, or transmitting the report of the MSI status to a healthcare provider.

Also disclosed herein are methods for detecting a deficient DNA mismatch repair mechanism in a sample from a subject, the methods comprising: receiving, by one or more processors, nucleic acid sequence data for a plurality of microsatellite loci in the sample; identifying, by the one or more processors, a set of microsatellite loci from the plurality of microsatellite loci based on a coverage requirement; applying, by the one or more processors, a set of sequence-based exclusion criteria to the set of microsatellite loci to identify a subset of the set of microsatellite loci; determining, by the one or more processors, a microsatellite instability (MSI) score for the sample based on a number of microsatellite loci in the set and a number of microsatellite loci in the subset; comparing, by the one or more processors, the MSI score to a threshold; determining an MSI status of high microsatellite instability for the sample if the MSI score is greater than or equal to the threshold; and using the determined MSI status to generate a genomic profile for the subject, wherein the MSI status of high microsatellite instability is indicative of a deficient DNA mismatch repair mechanism in a tissue of the subject.

In some embodiments, the sample is a tissue sample or a biopsy sample derived from the subject. In some embodiments, the sample is a liquid or hematological biopsy sample derived from the subject. In some embodiments, the sample is a liquid biopsy sample comprising blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some embodiments, the sample is a liquid biopsy sample or a hematological sample, and wherein the plurality of microsatellite loci comprises at least 1,000 loci. In some embodiments, the sample is a tissue sample or a biopsy sample, and wherein the plurality of microsatellite loci comprises at least 2,000 loci. In some embodiments, the microsatellite loci comprise alleles having mononucleotide, dinucleotide, or trinucleotide repeat sequences. In some embodiments, the sample is a tissue sample or a biopsy sample, and each microsatellite locus in the plurality of microsatellite loci comprises an allele having an overall length of at least 6 base pairs and less than 30 base pairs. In some embodiments, each microsatellite locus in the plurality of microsatellite loci comprises an allele having a mononucleotide, dinucleotide, or trinucleotide repeat sequence at a minimum of 5× repeats, and having a total length of less than 50 base pairs. In some embodiments, the nucleic acid sequence data is acquired using next generation sequencing (NGS), whole genome sequencing (WGS), whole exome sequencing, targeted or direct sequencing, or Sanger sequencing. In some embodiments, the coverage requirement is at least 75×, 100×, 150×, 150×, 200×, or 250×. In some embodiments, the coverage requirement is locus-dependent.

In some embodiments, applying the set of sequence-based exclusion criteria comprises excluding, from the set of microsatellite loci, any microsatellite locus that comprises an allele having an allele frequency below an allele frequency requirement. In some embodiments, the allele frequency requirement is at least 1%, at least 2%, at least 3%, at least 4%, at least 5%, at least 6%, at least 7%, at least 8%, at least 9%, or at least 10%. In some embodiments, applying the set of sequence-based exclusion criteria comprises excluding, from the set of microsatellite loci, any microsatellite locus that comprises an erroneous allele sequence according to a statistical model. In some embodiments, applying the set of sequence-based exclusion criteria comprises: comparing a particular allele at a particular microsatellite locus from the set of microsatellite loci to a reference database of sequencing errors; and excluding the particular microsatellite locus from the set of microsatellite loci if the particular allele corresponds to a known sequencing error.

In some embodiments, the particular microsatellite locus is excluded if the particular allele is an allele of less than 10 base pairs in length and the particular allele has an allele frequency less than or equal to a mean allele frequency plus two standard deviations for the particular allele in the reference database of sequencing errors. In some embodiments, the particular microsatellite locus is excluded if the particular allele is an allele of greater than or equal to 10 base pairs in length and the particular allele has an allele frequency less than or equal to a mean allele frequency plus three standard deviations for the particular allele in the reference database of sequencing errors.

In some embodiments, applying the set of sequence-based exclusion criteria comprises comparing a particular allele at a particular microsatellite locus to one or more databases; and excluding the particular of microsatellite locus if the particular allele corresponds to a known germline allele. In some embodiments, applying the set of sequence-based exclusion criteria comprises comparing a particular allele at a particular microsatellite locus to one or more databases; and excluding the particular microsatellite locus if the particular allele is equal in repeat length to a repeat length for the particular allele in the one or more databases, equal in overall length to an overall length for the particular allele in the reference human genome database, or equal in number of repeats to a number of repeats for the particular allele in the one or more databases. In some embodiments, the set of sequence-based exclusion criteria is locus-dependent.

In some embodiments, the MSI score is calculated by comparing the number of microsatellite loci in the subset to the number of microsatellite loci in the set.

In some embodiments, the MSI status is used to diagnose or confirm a diagnosis of disease in the subject. In some embodiments, the disease is cancer. In some embodiments, the cancer is bladder cancer, brain cancer, breast cancer, colorectal cancer, gastrointestinal cancer, kidney cancer, liver cancer, lung cancer, osteogenic carcinoma, ovarian cancer, pancreatic cancer, prostate cancer, rectal cancer, skin cancer, and uterine cancer, leukemia, lymphomas, and endometrial cancer. In some embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity, cancer of the pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera. Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma. Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. In some embodiments, the method further comprises selecting a cancer therapy to administer to the subject based on the MSI score. In some embodiments, the method further comprises determining an effective amount of a cancer therapy to administer to the subject based on the MSI score. In some embodiments, the method further comprises administering a cancer therapy to the subject based on the MSI status. In some embodiments, the cancer therapy comprises chemotherapy, radiation therapy, immunotherapy, surgery, or a therapy configured to target a defect in a DNA mismatch repair (MMR) pathway. In some embodiments, the cancer is colorectal cancer (CRC), prostate cancer, leukemia, bladder cancer, ovarian cancer, endometrial cancer, pancreatic ductal adenocarcinoma, or follicular thyroid cancer, and the cancer therapy comprises an anti-programmed death-1 (anti-PD-1) or anti-programmed death ligand-1 (anti-PD-L1) therapy. In some embodiments, the cancer is gastric cancer, and the cancer therapy comprises performing a surgical resection.

In some embodiments, the threshold is a first threshold, and the method further comprises: comparing the MSI score to a second threshold if the MSI score is less than the first threshold; and calling an MSI status of microsatellite stable for the sample if the MSI score is less than or equal to the second threshold; or calling an MSI status of equivocal microsatellite instability for the sample if the MSI score is greater than the second threshold.

In some embodiments, the first threshold is determined by: performing a plurality of iterations to obtain a plurality of candidate first threshold values, and averaging the plurality of candidate first threshold values to determine the first threshold. In some embodiments, each iteration comprises: randomly selecting a subset of a plurality of samples from a plurality of patients; calculating a plurality of MSI scores for the subset; and obtaining a candidate first threshold value that maximizes concordance with determinations of high microsatellite instability obtained using a reference microsatellite instability assay for the subset of the plurality of samples from the plurality of patients. In some embodiments, the candidate first threshold value is set to a value that maximizes a sum of positive percent agreement (PPA) and negative percent agreement (NPA) with microsatellite instability status results obtained using the reference microsatellite instability assay, while requiring that NPA is greater than a first minimum requirement.

In some embodiments, the second threshold is determined by: performing a plurality of iterations to obtain a plurality of candidate second threshold values, and averaging the plurality of candidate second threshold values to determine the second threshold. In some embodiments, each iteration comprises: randomly selecting a subset of a plurality of samples from a plurality of patients; calculating a plurality of MSI scores for the subset; and obtaining a candidate second threshold value that maximizes concordance with determinations of microsatellite stability obtained using a reference microsatellite instability assay for the subset of the plurality of samples from the plurality of patients. In some embodiments, the candidate second threshold value is set to a value that maximizes the sum of positive percent agreement (PPA) and negative percent agreement (NPA) with microsatellite instability status results obtained using the reference microsatellite instability assay, while requiring that NPA is greater than a second minimum requirement.

In some embodiments, the method further comprises generating a report of the MSI status. In some embodiments, the method further comprises displaying the report of the MSI status on a display device. In some embodiments, the method further comprises transmitting the report of the MSI status to a healthcare provider. In some embodiments, the report is transmitted over the Internet or via a peer-to-peer connection.

Disclosed herein are methods of selecting a cancer therapy, the methods comprising: responsive to determining a microsatellite instability status for a sample from a subject, selecting a cancer therapy for the subject, wherein the microsatellite instability status is determined according to any of the methods disclosed herein.

Disclosed herein are methods of treating a cancer in a subject, comprising: responsive to determining a microsatellite instability status for a sample from the subject, administering an effective amount of a cancer therapy to the subject, wherein the microsatellite instability status is determined according to any of the methods disclosed herein.

In some embodiments, the cancer therapy is for treating bladder cancer, brain cancer, breast cancer, colorectal cancer, gastrointestinal cancer, kidney cancer, liver cancer, lung cancer, osteogenic carcinoma, ovarian cancer, pancreatic cancer, prostate cancer, rectal cancer, skin cancer, and uterine cancer, leukemia, lymphomas, and endometrial cancer. In some embodiments, the cancer therapy comprises a therapy that targets a defect in a DNA mismatch repair (MMR) pathway. In some embodiments, the cancer therapy comprises chemotherapy, radiation therapy, immunotherapy, surgery, or a therapy configured to target a defect in a DNA mismatch repair (MMR) pathway. In some embodiments, the cancer is colorectal cancer (CRC), prostate cancer, leukemia, bladder cancer, ovarian cancer, endometrial cancer, pancreatic ductal adenocarcinoma, or follicular thyroid cancer, the microsatellite instability status is high, and the cancer therapy comprises an anti-programmed death-1 (anti-PD-1) or anti-programmed death ligand-1 (anti-PD-L1) therapy. In some embodiments, the cancer is gastric cancer, the microsatellite instability status is high, and the cancer therapy comprises performing a surgical resection.

Disclosed herein are methods for monitoring tumor progression or recurrence in a subject, the methods comprising: determining a first microsatellite instability status in a first sample obtained from the subject at a first time point according to any of the method disclosed herein; determining a second microsatellite instability status in a second sample obtained from the subject at a second time point; and comparing the first microsatellite stability status to the second microsatellite stability status, thereby monitoring the tumor progression or recurrence.

In some embodiments, the second microsatellite instability status for the second sample is determined according to any of the methods disclosed herein. In some embodiments, the method further comprises adjusting a tumor therapy in response to the tumor progression. In some embodiments, the method further comprises adjusting a dosage of the tumor therapy or selecting a different tumor therapy in response to the tumor progression. In some embodiments, the method further comprises administering the adjusted tumor therapy to the subject. In some embodiments, the first time point is before the subject has been administered a tumor therapy, and wherein the second time point is after the subject has been administered the tumor therapy. In some embodiments, the subject has a cancer, is at risk of having a cancer, or is suspected of having a cancer. In some embodiments, the cancer is a solid tumor. In some embodiments, the cancer is a hematological cancer.

Also disclosed herein are systems comprising: one or more processors; a memory storing one or more programs configured to be executed by the one or more processors, the one or more programs including instructions for: receiving, by one or more processors, nucleic acid sequence data for a plurality of microsatellite loci in a sample; identifying, by the one or more processors, a set of microsatellite loci from the plurality of microsatellite loci based on a coverage requirement; applying, by the one or more processors, a set of sequence-based exclusion criteria to the set of microsatellite loci to identify, a subset of the set of microsatellite loci; determining, by the one or more processors, a microsatellite instability (MSI) score for the sample based on a number of microsatellite loci in the set and a number of microsatellite loci in the subset; comparing, by the one or more processors, the MSI score to a threshold; determining an MSI status of high microsatellite instability for the sample if the MSI score is greater than or equal to the threshold, and using the determined MSI status to generate a genomic profile for a subject from which the sample was derived, wherein the MSI status of high microsatellite instability is indicative of a deficient DNA mismatch repair mechanism in the sample. In some embodiments, the threshold is determined by: performing a plurality of iterations to obtain a plurality of candidate first threshold values; and averaging the plurality of candidate first threshold values to determine the first threshold. In some embodiments, each iteration comprises: randomly selecting a subset of a plurality of samples from a plurality of patients; calculating a plurality of MSI scores for the subset; and obtaining a candidate first threshold value that maximizes concordance with determinations of high microsatellite instability obtained using a reference microsatellite instability assay for the subset of the plurality of samples from the plurality of patients. In some embodiments, the candidate first threshold value is set to a value that maximizes a sum of positive percent agreement (PPA) and negative percent agreement (NPA) with microsatellite instability status results obtained using the reference microsatellite instability assay, while requiring that NPA is greater than a first minimum requirement. In some embodiments, the threshold is a first threshold, and the one or more programs further comprise instructions for: comparing the MSI score to a second threshold if the MSI score is less than the first threshold; and calling an MSI status of microsatellite stable for the sample if the MSI score is less than or equal to the second threshold; or calling an MSI status of equivocal microsatellite instability for the sample if the MST score is greater than the second threshold. In some embodiments, the second threshold is determined by: performing a plurality of iterations to obtain a plurality of candidate second threshold values; and averaging the plurality of candidate second threshold values to determine the second threshold. In some embodiments, each iteration comprises: randomly selecting a subset of a plurality of samples from a plurality of patients; calculating a plurality of MSI scores for the subset; and obtaining a candidate second threshold value that maximizes concordance with determinations of microsatellite stability obtained using a reference microsatellite instability assay for the subset of the plurality of samples from the plurality of patients. In some embodiments, the candidate second threshold value is set to a value that maximizes the sum of positive percent agreement (PPA) and negative percent agreement (NPA) with microsatellite instability status results obtained using the reference microsatellite instability assay, while requiring that NPA is greater than a second minimum requirement.

Disclosed herein are non-transitory computer-readable storage media storing one or more programs, the one or more programs comprising instructions, which when executed by one or more processors of an electronic device or system, cause the electronic device or system to: receive, by one or more processors, nucleic acid sequence data for a plurality of microsatellite loci in a sample; identify, by the one or more processors, a set of microsatellite loci from the plurality of microsatellite loci based on a coverage requirement; apply, by the one or more processors, a set of sequence-based exclusion criteria to the set of microsatellite loci to identify a subset of the set of microsatellite loci; determine, by the one or more processors, a microsatellite instability (MSI) score for the sample based on a number of microsatellite loci in the set and a number of microsatellite loci in the subset; compare, by the one or more processors, the MST score to a threshold; determine an MSI status of high microsatellite instability for the sample if the MSI score is greater than or equal to the threshold, and use the determined MSI status to generate a genomic profile for a subject from which the sample was derived, wherein the MSI status of high microsatellite instability is indicative of a deficient DNA mismatch repair mechanism in the sample. In some embodiments, the threshold is a first threshold, and the one or more programs further comprise instructions for: comparing the MSI score to a second threshold if the MSI score is less than the first threshold; and calling an MSI status of microsatellite stable for the sample if the MSI score is less than or equal to the second threshold; or calling an MSI status of equivocal microsatellite instability for the sample if the MSI score is greater than the second threshold.

In some embodiments, the coverage requirement is at least 75×, 100×, 150×, 150×, 200×, or 250×. In some embodiments, the coverage requirement is locus-dependent. In some embodiments, applying the set of sequence-based exclusion criteria comprises excluding, from the set of microsatellite loci, a microsatellite locus that comprises an allele having an allele frequency below an allele frequency requirement. In some embodiments, the allele frequency requirement is at least 1%, at least 2%, at least 3%, at least 4%, at least 5%, at least 6%, at least 7%, at least 8%, at least 9%, or at least 10%. In some embodiments, applying the set of sequence-based exclusion criteria comprises excluding, from the set of microsatellite loci, a microsatellite locus that comprises an erroneous allele sequence according to a statistical model. In some embodiments, applying the set of sequence-based exclusion criteria comprises: comparing a particular allele at a particular microsatellite locus from the set of microsatellite loci to a reference database of sequencing errors; and excluding the particular microsatellite locus from the set of microsatellite loci if the particular allele corresponds to a known sequencing error. In some embodiments, the particular microsatellite locus is excluded if the particular allele is an allele of less than 10 base pairs in length and the particular allele has an allele frequency less than or equal to a mean allele frequency plus two standard deviations for the particular allele in the reference database of sequencing errors. In some embodiments, the particular microsatellite locus is excluded if the particular allele is an allele of greater than or equal to 10 base pairs in length and the particular allele has an allele frequency less than or equal to a mean allele frequency plus three standard deviations for the particular allele in the reference database of sequencing errors. In some embodiments, applying the set of sequence-based exclusion criteria comprises comparing a particular allele at a particular microsatellite locus to one or more databases; and excluding the particular of microsatellite locus if the particular allele corresponds to a known germline allele. In some embodiments, applying the set of sequence-based exclusion criteria comprises comparing a particular allele at a particular microsatellite locus to one or more databases; and excluding the particular microsatellite locus if the particular allele is equal in repeat length to a repeat length for the particular allele in the reference human genome database, equal in overall length to an overall length for the particular allele in the reference human genome database, or equal in number of repeats to a number of repeats for the particular allele in the reference human genome database. In some embodiments, the set of sequence-based exclusion criteria is locus-dependent.

In some embodiments of any of the methods disclosed herein, the method further comprises displaying a user interface comprising the MSI status via an online portal. In some embodiments, the method further comprises displaying a user interface comprising the MSI status via a mobile device. In some embodiments, the user interface comprises an MSI score data structure field. In some embodiments, the method further comprises determining, identifying, or applying the MSI status of the sample as a diagnostic value associated with the sample. In some embodiments, the method further comprises generating a genomic profile for the subject based on the MSI status. In some embodiments, the method further comprises administering an anti-cancer agent or applying an anti-cancer treatment to the subject based on the generated genomic profile. In some embodiments, the MSI status of the sample is used in making suggested treatment decisions for the subject. In some embodiments, the MSI status of the sample is used in applying or administering a treatment to the subject.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1A and FIG. 1B provide non-limiting examples of a workflow for determining the microsatellite instability (MSI) status of a sample according to the methods described herein.

FIG. 2 provides a second non-limiting example of a workflow for determining the microsatellite instability (MSI) status of a sample according the methods described herein.

FIG. 3 provides a non-limiting example of a process used to determine a threshold for distinguishing between microsatellite instability—high (MSI-H) samples and other samples based on a microsatellite instability (MSI) score.

FIG. 4 provides a non-limiting example of a process used to determine a threshold for distinguishing between microsatellite stable (MSS) samples and other samples based on a microsatellite instability (MSI) score.

FIG. 5 provides a non-limiting schematic illustration of an electronic device or computer system according to examples of the present disclosure.

FIG. 6 provides a non-limiting schematic illustration of a computer network according to examples of the present disclosure.

FIG. 7 provides a non-limiting example of data for the fraction of loci that were determined to be unstable (i.e., a “fraction unstable” or MSI score) in clinical samples versus the number of microsatellite loci evaluated and used to calculate the fraction unstable score.

FIGS. 8A-8D provide non-limiting examples of concordance data for MSI scores computed according to the disclosed methods with those obtained using a reference method as a function of different numbers of microsatellite loci used for the evaluation. FIG. 8A shows a concordance plot of MSI scores comprising data for all available/evaluable loci for the sample.

FIG. 8B shows a concordance plot of MSI scores comprising data for 1,500 microsatellite loci.

FIG. 8C shows a concordance plot of MSI scores comprising data for 1,000 microsatellite loci.

FIG. 8D shows a concordance plot of MSI scores comprising data for 500 microsatellite loci.

FIG. 9 provides a non-limiting example of data for the fraction unstable score plotted as a function of the amount of DNA from a known microsatellite unstable sample mixed into normal DNA and used to determine a limit-of-detection (LOD) for the disclosed methods.

FIG. 10 provides a non-limiting example of data for fraction unstable score plotted for a series of negative control samples.

FIG. 11 provides a non-limiting example of data for fraction unstable score plotted for microsatellite instability—high (MSI-H) samples and normal samples.

FIG. 12 provides a non-limiting example of data for MSI scores calculated for a series of samples that demonstrates the robustness of the disclosed methods to confounding effects of, for example, poor quality reagents used during the sample preparation and sequencing processes used to generate sequence data for selected microsatellite loci.

FIG. 13 provides a non-limiting example of fraction unstable scores for clinical samples grouped by disease type.

FIG. 14 provides a non-limiting example of the output provided by a system for determining microsatellite instability according to the present disclosure.

FIG. 15 provides a non-limiting example of the output provided by a system for determining microsatellite instability according to the present disclosure.

### DETAILED DESCRIPTION

Methods and systems for evaluating microsatellite instability (MSI) status of a sample are described. MSI can be defined as any change in allele length due to either insertion or deletion of repeating units in a microsatellite within a tumor when compared to normal tissue (Boland, et al. (1998), “A National Cancer Institute Workshop on Microsatellite Instability for Cancer Detection and Familial Predisposition: Development of Intemational Criteria for the Determination of Microsatellite Instability in Colorectal Cancer”, Cancer Res. 58:5248-5257). Detection of MSI thus provides phenotypic evidence that MMR is not functioning normally in cells (e.g., due to an inactivating mutation or epigenetic silencing of one or more of the MMR pathway genes, such as MSH2, MSH6, MLH1, and PMS2 (Hempelmann, et al. (2018), “Microsatellite instability in prostate cancer by PCR or next-generation sequencing”, Journal for ImmunoTherapy of Cancer 6:29)), and provides a potential biomarker for the detection of cancer, for diagnosis of cancer, for disease prognosis, for selection of a cancer therapy, and/or for monitoring tumor progression in a subject (e.g., a patient) for which MSI has been detected in a tumor tissue sample. Microsatellite instability is found most often in colorectal cancer, gastric cancer, and endometrial cancer, but may be found in many other types of cancer as well.

The disclosed methods and systems utilize nucleic acid sequencing data and a per-locus analysis of variant allele frequencies (i.e., the frequencies for variant alleles of altered length) at a plurality of microsatellite loci. Several filters are used to exclude candidate alleles that are likely to be the result of noise, sequencing errors, or that are germline alleles. The input microsatellite allele sequences are individually categorized as stable or unstable, and an MSI score for the sample is calculated by dividing the number of unstable loci (i.e., loci exhibiting at least one unstable allele) by the total number of loci evaluated for allelic stability (e.g., the total number of loci that met a specified minimum sequencing coverage requirement) to determine the fraction of microsatellite loci that are unstable. A threshold is then applied to the MSI score for classification of the sample as microsatellite instability—high (MSI-H), microsatellite instability—equivocal (MSI-E), or microsatellite stable (MSS).

The disclosed methods exhibit increased sensitivity and accuracy compared to PCR-based methods or existing sequencing-based methods for determining MSI status due to the large number of microsatellite loci that may be interrogated and the enforcement of minimum sequencing coverage requirements and minimum microsatellite allele frequency requirements. The methods exhibit reduced sensitivity to germline variation, improved robustness against confounding effects, an improved limit of detection (LoD), and eliminate the need for a matched normal sample while taking advantage of automated or semi-automated sequencing analysis processes to reduce analysis cost and improve inter-laboratory variability.

In some instances, the disclosed methods for determining microsatellite instability status may be implemented as part of a genomic profiling process that comprises, identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for determining microsatellite instability as part of a genomic profiling process can improve the validity of, e.g., disease detection calls, made on the basis of the genomic profiling by, for example, independently confirming the presence of an impaired DNA mismatch repair (MMR) mechanism in a given patient sample. In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, for example, the disclosed methods for detecting a deficient DNA mismatch repair mechanism and/or for evaluating microsatellite instability in a sample from a subject may comprise: receiving, by one or more processors, nucleic acid sequence data for a plurality of microsatellite loci in the sample; identifying, by the one or more processors, a set of microsatellite loci from the plurality of microsatellite loci based on a coverage requirement; applying, by the one or more processors, a set of sequence-based exclusion criteria to the set of microsatellite loci to identify a subset of the set of microsatellite loci: calculating, by the one or more processors, a microsatellite instability (MSI) score for the sample based on the number of microsatellite loci in the set and the number of microsatellite loci in the subset; comparing, by the one or more processors, the MSI score to a threshold (e.g., a first threshold); and if the MSI score is greater than or equal to the threshold, determining an MSI status of high microsatellite instability (MSI-H) for the sample, wherein the MSI status of high microsatellite instability is indicative of a deficient DNA mismatch repair mechanism in a tissue of the subject. As used herein. “applying” a set of exclusion criteria to a set of microsatellite loci may comprise “filtering” the set of microsatellite loci, or “removing” microsatellite loci that meet the set of exclusion criteria from the original set of microsatellite loci.

In some instances, the disclosed methods may further comprise comparing the MSI score to a second threshold if the MSI score is less than the first (e.g., the predetermined) threshold; and if the MSI score is less than or equal to the second threshold, determining an MSI status of microsatellite stable (MSS) for the sample; if the MSI score is greater than the second threshold, determining an MSI status of equivocal microsatellite instability (MSI-E) for the sample.

In some instances, the threshold (or first threshold) may be determined by performing a plurality of iterations to obtain a plurality of candidate first threshold values, and averaging the plurality of candidate first threshold values to determine the first threshold. In some instances, each iteration of the (or first) threshold determination process comprises: randomly selecting a subset of a plurality of samples from a plurality of patients; calculating a plurality of MSI scores for the subset; and obtaining a candidate first threshold value that maximizes concordance with determinations of high microsatellite instability obtained using a reference microsatellite instability assay for the subset of the plurality of samples from the plurality of patients. In some instances, for example, the candidate first threshold value is set to a value that maximizes a sum of positive percent agreement (PPA) and negative percent agreement (NPA) with microsatellite instability status results obtained using the reference microsatellite instability assay, while requiring that NPA is greater than a first minimum requirement.

In some instances, the second threshold is determined by performing a plurality of iterations to obtain a plurality of candidate second threshold values, and averaging the plurality of candidate second threshold values to determine the second threshold. In some instances, each iteration of the second threshold determination process may comprises randomly selecting a subset of a plurality of samples from a plurality of patients; calculating a plurality of MSI scores for the subset; and obtaining a candidate second threshold value that maximizes concordance with determinations of microsatellite stability obtained using a reference microsatellite instability assay for the subset of the plurality of samples from the plurality of patients. In some instances, the candidate second threshold value is set to a value that maximizes the sum of positive percent agreement (PPA) and negative percent agreement (NPA) with microsatellite instability status results obtained using the reference microsatellite instability assay, while requiring that NPA is greater than a second minimum requirement.

The disclosed methods and systems may be applied to the analysis of any of a variety of samples (for example, tissue samples, biopsy samples, blood samples, urine samples, saliva samples, or liquid biopsy samples) derived from a subject, and from which nucleic acid molecules (e.g., genomic DNA. mRNA, etc.) may be extracted and sequenced. The sequencing-based methods described herein enable the analysis of a large number of microsatellite loci (e.g., hundreds to thousands of individual microsatellite loci)—each comprising mononucleotide, dinucleotide, trinucleotide, or longer repeat sequence motifs—for the presence of variant alleles having altered length. Various exclusion criteria may be applied to the input microsatellite sequence data, for example, to eliminate loci for which the sequencing coverage is inadequate, or to eliminate loci that exhibit alleles that fail to meet a minimum allele frequency requirement, correspond to known germline alleles, correspond to known sequencing errors, and the like, from the analysis, thereby improving the accuracy of the determination of microsatellite instability (MSI) status as a biomarker for the detection of cancer, for providing a disease prognosis, for selection of a cancer therapy, and/or for monitoring tumor progression in a patient.

Also disclosed herein are methods and systems for detecting a cancer, diagnosing a cancer, providing a disease prognosis, selecting a cancer therapy, and/or monitoring tumor progression in a patient, wherein the methods may comprise: receiving, by one or more processors, nucleic acid sequence data for a plurality of microsatellite loci in the sample; identifying, by the one or more processors, a set of microsatellite loci from the plurality of microsatellite loci based on a coverage requirement; applying, by the one or more processors, a set of sequence-based exclusion criteria to the set of microsatellite loci to identify a subset of the set of microsatellite loci; calculating, by the one or more processors, a microsatellite instability (MSI) score for the sample based on the number of microsatellite loci in the set and the number of microsatellite loci in the subset; comparing, by the one or more processors, the MSI score to a threshold (e.g., a first threshold); and if the MSI score is greater than or equal to the threshold, determining an MSI status of high microsatellite instability (MSI-H) for the sample, wherein the MSI status of high microsatellite instability is indicative of a deficient DNA mismatch repair mechanism in a tissue of the subject. In some instances, the methods may further comprise comparing the MSI score to a second threshold if the MSI score is less than the first (e.g., the predetermined) threshold; and if the MSI score is less than or equal to the second threshold, determining an MSI status of microsatellite stable (MSS) for the sample; if the MSI score is greater than the second threshold, determining an MSI status of equivocal microsatellite instability (MSI-E) for the sample.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the term “subgenomic interval” (or “subgenomic sequence interval”) refers to a portion of genomic sequence. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., an entire gene, or a portion thereof. e.g., the coding region (or portions thereof), an intron (or portion thereof), exon (or portion thereof), or microsatellite region (or portion thereof). A subgenomic interval can comprise all or a part of a fragment of a naturally occurring, e.g., genomic DNA, nucleic acid. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

As used herein, the term “subject interval” refers to a subgenomic sequence interval or an expressed subgenomic sequence interval (e.g., the transcribed sequence of a subgenomic interval). In some instances, a subgenomic sequence interval and an expressed subgenomic sequence interval correspond, meaning that the expressed subgenomic sequence interval comprises a sequence expressed from the corresponding subgenomic sequence interval. In some instances, a subgenomic sequence interval and an expressed subgenomic sequence interval are non-corresponding, meaning that the expressed subgenomic sequence interval does not comprise a sequence expressed from the non-corresponding subgenomic sequence interval, but rather corresponds to a different subgenomic sequence interval. In some instances, a subgenomic sequence interval and an expressed subgenomic sequence interval partially correspond, meaning that the expressed subgenomic sequence interval comprises a sequence expressed from the corresponding subgenomic sequence interval and a sequence expressed from a different corresponding subgenomic sequence interval.

The terms “cancer” and “cancerous” refer to or describe the physiological condition in mammals that is typically characterized by unregulated cell growth. Included in this definition are benign and malignant cancers. By “early stage cancer” or “early stage tumor” is meant a cancer that is not invasive or metastatic or is classified as a Stage 0, 1, or 2 cancer. Examples of a cancer include, but are not limited to, a lung cancer (e.g., a non-small cell lung cancer (NSCLC)), a kidney cancer (e.g., a kidney urothelial carcinoma), a bladder cancer (e.g., a bladder urothelial (transitional cell) carcinoma), a breast cancer, a colorectal cancer (e.g., a colon adenocarcinoma), an ovarian cancer, a pancreatic cancer, a gastric carcinoma, an esophageal cancer, a mesothelioma, a melanoma (e.g., a skin melanoma), a head and neck cancer (e.g., a head and neck squamous cell carcinoma (HNSCC)), a thyroid cancer, a sarcoma (e.g., a soft-tissue sarcoma, a fibrosarcoma, a myxosarcoma, a liposarcoma, an osteogenic sarcoma, an osteosarcoma, a chondrosarcoma, an angiosarcoma, an endotheliosarcoma, a lymphangiosarcoma, a lymphangioendotheliosarcoma, a leiomyosarcoma, or a rhabdomyosarcoma), a prostate cancer, a glioblastoma, a cervical cancer, a thymic carcinoma, a leukemia (e.g., an acute lymphocytic leukemia (ALL), an acute myelocytic leukemia (AML), a chronic myelocytic leukemia (CML), a chronic eosinophilic leukemia, or a chronic lymphocytic leukemia (CLL)), a lymphoma (e.g., a Hodgkin lymphoma or a non-Hodgkin lymphoma (NHL)), a myeloma (e.g., a multiple myeloma (MM)), a mycoses fungoides, a merkel cell cancer, a hematologic malignancy (e.g., leukemia, lymphoma, or multiple myeloma), a cancer of hematological tissues, a B cell cancer, a bronchus cancer, a stomach cancer, a brain or central nervous system cancer, a peripheral nervous system cancer, a uterine or endometrial cancer, a cancer of the oral cavity or pharynx, a liver cancer, a testicular cancer, a biliary tract cancer, a small bowel or appendix cancer, a salivary gland cancer, an adrenal gland cancer, an adenocarcinoma, an inflammatory myofibroblastic tumor, a gastrointestinal stromal tumor (GIST), a colon cancer, a myelodysplastic syndrome (MDS), a myeloproliferative disorder (MPD), a polycythemia Vera, a chordoma, a synovioma, an Ewing's tumor, a squamous cell carcinoma, a basal cell carcinoma, an adenocarcinoma, a sweat gland carcinoma, a sebaceous gland carcinoma, a papillary carcinoma, a papillary adenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilms' tumor, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a meningioma, a neuroblastoma, a retinoblastoma, a follicular lymphoma, a diffuse large B-cell lymphoma, a mantle cell lymphoma, a hepatocellular carcinoma, a thyroid cancer, a small cell cancer, an essential thrombocythemia, an agnogenic myeloid metaplasia, a hypereosinophilic syndrome, a systemic mastocytosis, a familiar hypereosinophilia, a neuroendocrine cancer, or a carcinoid tumor.

The term “tumor,” as used herein, refers to all neoplastic cell growth and proliferation, whether malignant or benign, and all pre-cancerous and cancerous cells and tissues. The terms “cancer,” “cancerous,” and “tumor” are not mutually exclusive as referred to herein.

The section headings used herein are for organizational purposes only and are not to be construed as limiting the subject matter described.

### Methods for Determining Microsatellite Instability (MSI) Status:

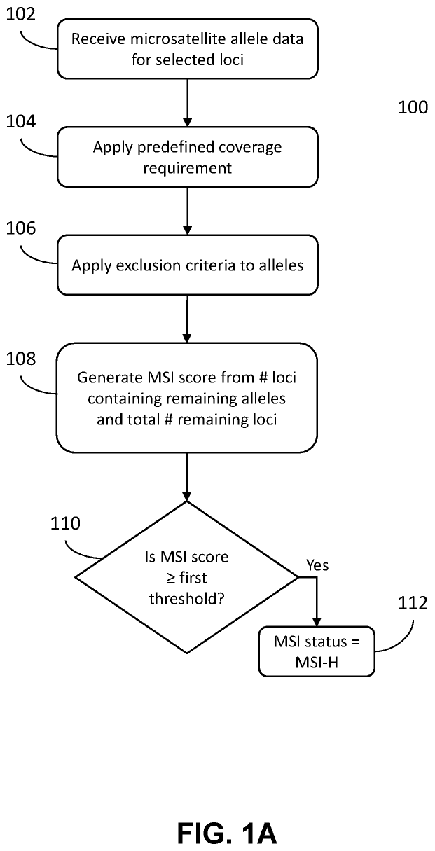

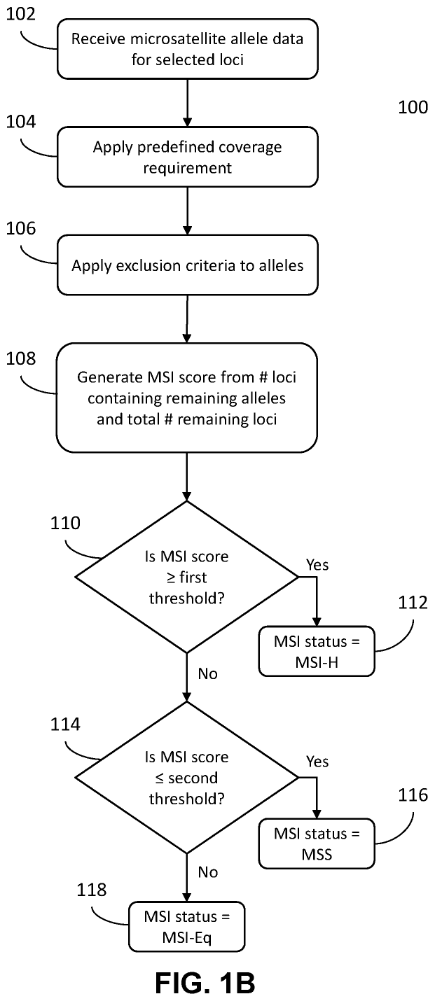

FIG. 1A provides a non-limiting example of a process 100 for determining the microsatellite instability (MSI) status of a sample according to the methods disclosed herein. Nucleic acid sequencing data for microsatellite alleles at a plurality of selected loci are received at step 102. At step 104, a coverage requirement is applied to the received data to, for example, exclude loci for which sequencing coverage fails to meet a defined minimum coverage threshold from further analysis. The sequencing data may be further processed by applying a set of exclusion criteria at step 106 to, for example, exclude loci that exhibit alleles that fail to meet a minimum allele frequency requirement, exclude loci that exhibit alleles that correspond to known germline alleles, exclude loci that correspond to known sequencing errors, and the like, from further analysis, thereby improving the accuracy of the determination of microsatellite instability (MSI) status as a biomarker. A microsatellite instability (MSI) score (corresponding to the fraction of total remaining loci that exhibit unstable alleles) is then calculated at step 108 for the sample by dividing the number of unstable loci detected (i.e., the number of loci exhibiting at least one unstable allele at step 106) by the total number of loci evaluated for allelic stability (i.e., the total number of loci evaluated at step 104). Comparison of the MSI score to a first cutoff threshold at step 110 (e.g., a predetermined threshold) is used to determine if the sample exhibits a microsatellite instability—high (MSI-H) status, which may be output at step 112. High microsatellite instability can be an indicator of, for example, a deficient DNA mismatch repair mechanism in a tissue sample collected from a subject (e.g., a patient).

In some instances, as illustrated in FIG. 1B, if the MSI score is less than the first cutoff threshold, the score is compared to a second cutoff threshold at step 114. If the MSI score is less than or equal to the second cutoff threshold, a status of microsatellite stable (MSS) is assigned for the sample, which may be output at step 116. If the MST score is less than the first threshold and greater than the second threshold, a status of microsatellite instability—equivocal (MSI-E) is assigned for the sample, which may be output at step 118. In some instances, the MSI score and assignment of MST status for the sample are performed without having or requiring analysis of a paired “normal” sample.

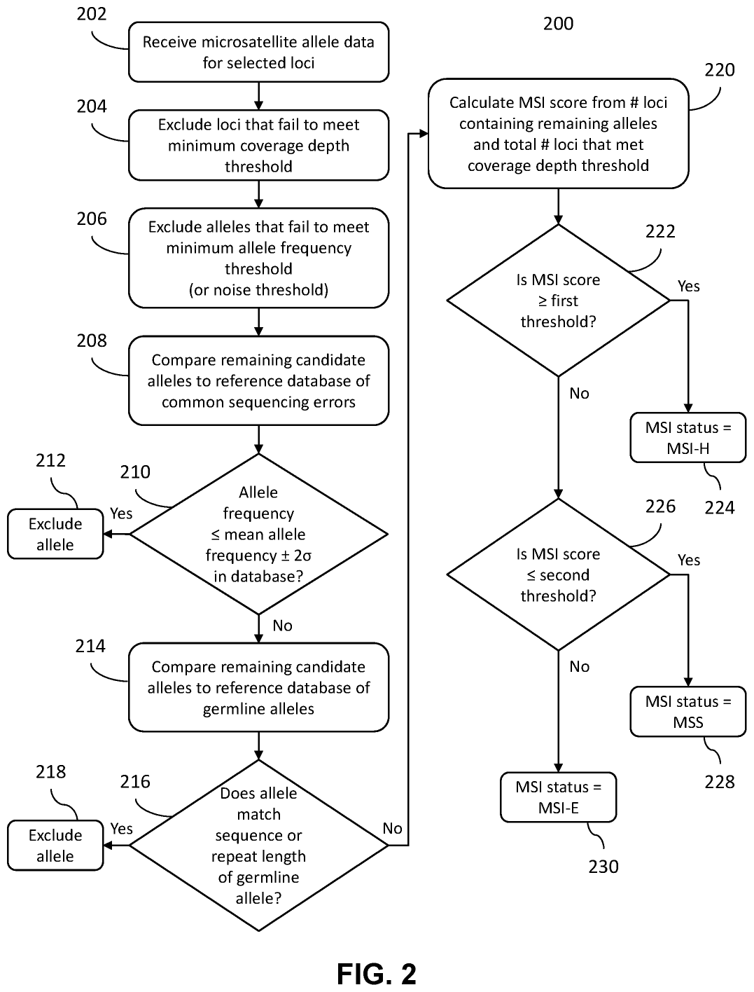

FIG. 2 provides a second non-limiting example of a process 200 for determining the microsatellite instability (MSI) status of a sample according the methods disclosed herein. Nucleic acid sequencing data for microsatellite alleles at a plurality of selected loci are received at step 202 and a minimum coverage depth threshold is applied at step 204 to exclude loci for which sequencing coverage fails to meet a defined minimum threshold (an example of the coverage requirement as illustrated in step 104 of FIG. 1A) from further analysis. For example, in some instances, the coverage requirement (step 104 in FIG. 1A) or minimum coverage depth (step 204 in FIG. 2) may be at least 75×, 100×, 150×, 150×, 200×, or 250×. The sequencing data may then be further processed by applying a set of sequence-based exclusion criteria at steps 206-216. At step 206, loci that exhibit alleles that fail to meet a minimum allele frequency requirement (e.g., a noise threshold) are excluded. The remaining candidate alleles may be compared to one or more reference databases (e.g., a single database, multiple databases, distributed databases) at step 208 to identify alleles that correspond to common sequencing errors. For example, if the observed allele frequency for a given locus is less than or equal to the mean allele frequency plus twice the standard deviation for that allele in the reference database(s) (e.g., the HG19 standard reference database and/or an internally developed reference database) at step 210, that locus is excluded from further analysis at step 212. In some instances, e.g., for repeat sequence motifs that are at least 8, 9, 10, 11, 12, or more than 12 bases in length, a locus may be excluded from further analysis at step 212 if the observed allele frequency for the locus is less than or equal to the mean allele frequency plus three times the standard deviation for that allele in the reference database(s) at step 210. All remaining candidate alleles are then compared to known germline alleles included in the reference database(s) at step 214. If a candidate allele matches a sequence or repeat length of a known germline allele, at step 218, the corresponding locus is excluded from further analysis.

In some embodiments, applying the set of sequence-based exclusion criteria comprises excluding, from the set of microsatellite loci, any microsatellite locus that comprises an erroneous allele sequence according to a statistical model. For example, the system performs a statistical assessment of the supporting read data to determine whether a given allele is present at a level significantly above the expected background level.

A microsatellite instability (MSI) score (corresponding to the fraction of total remaining loci that exhibit unstable alleles) is then calculated at step 220 for the sample by comparing the number of unstable loci detected (i.e., the number of loci exhibiting at least one unstable allele) to the total number of loci evaluated for allelic stability (i.e., the total number of loci that met the minimum coverage depth requirement at step 204). In some embodiments, the score is indicative of the degree to which the number of unstable loci is greater than expected for a normal sample. In some embodiments, the score is a ratio of the number of unstable loci detected (i.e., the number of loci exhibiting at least one unstable allele) to the total number of loci evaluated for allelic stability (i.e., the total number of loci that met the minimum coverage depth requirement at step 204). Comparison of the MSI score to a first cutoff threshold at step 222 (e.g., a predetermined threshold) is used to determine if the sample exhibits a microsatellite instability—high (MSI-H) status, which may be output at step 224.

In some instances, if the MSI score is less than the first cutoff threshold, the score is compared to a second cutoff threshold at step 226. If the MSI score is less than or equal to the second cutoff threshold, a status of microsatellite stable (MSS) is assigned for the sample, which may be output at step 228. If the MSI score is less than the first threshold and greater than the second threshold, a status of microsatellite instability—equivocal (MSI-E) is assigned for the sample, which may be output at 230. In some instances, the MSI score and assignment of MSI status for the sample are performed without having or requiring analysis of a paired “normal” sample.

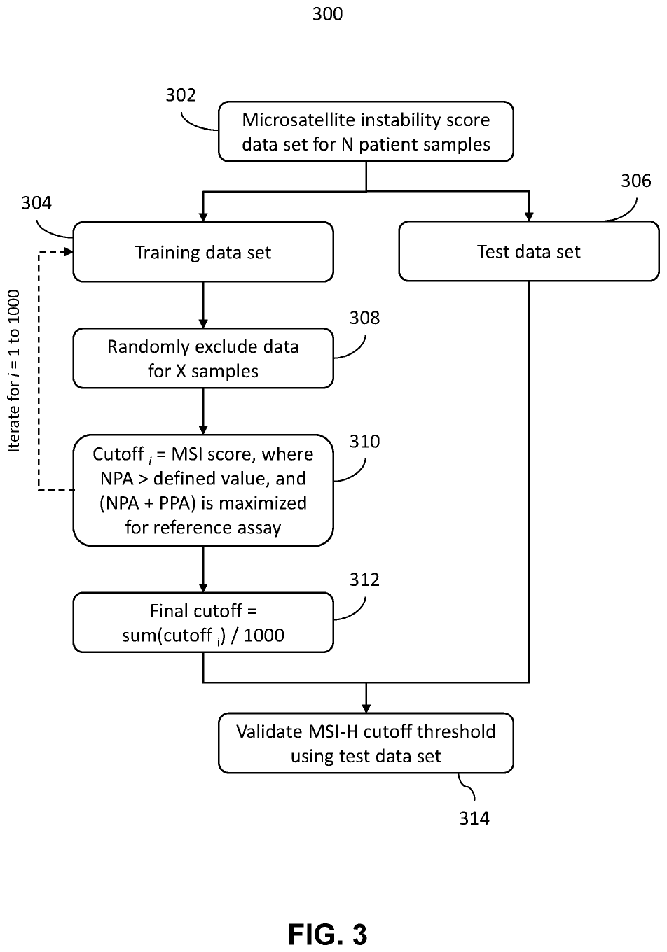

FIG. 3 provides a non-limiting example of a process 300 used to determine a threshold for distinguishing between microsatellite instability—high (MSI-H) samples and other samples based on a microsatellite instability (MSI) score. Microsatellite instability (MSI) score data for a plurality of patient samples, 302, is divided into a training data set, 304, and a test data set, 306, where the data split is stratified by MSI status as determined by a reference method and by cancer type(s) to ensure balanced sets of training and test data (e.g., so that the training and test data sets contain equivalent mixes of data for different cancer types and MSI status). In each iteration of the threshold determination process, at step 308, a subset of the training data is created by randomly excluding, e.g., 1%, 2%, 3%, 4%, 5%, 6%, 7%, 8%, 9%, 10%, or more than 10% of the samples, and, at step 310, a candidate cutoff threshold value is determined for the remaining data in the subset that maximizes the concordance of the training data subset with the results (e.g., the microsatellite instability—high (MSI-H) results) obtained using a reference assay for microsatellite instability (e.g., the Promega Microsatellite Instability (MSI) Analysis assay, Promega Corporation, Madison, WI). For example, the candidate cutoff threshold value for the training data subset may be adjusted to maximize concordance with the results of the reference assay by requiring that negative percent agreement (NPA) be greater than a defined value (e.g., 85%, 86%, 87%, 88%, 89%, 90%, 91%, 92%, 93%, 94%, 95%, 96%, 97%, 98%, or 99%) while the sum of positive percent agreement (PPA) and negative percent agreement (NPA) be maximized. A series of candidate cutoff threshold values are determined by iterating through the steps of defining a training data subset by randomly excluding some of the training data at step 308 and determining a cutoff threshold value that maximizes concordance with the reference assay at step 310, where the steps are repeated for, e.g., at least 100, 200, 300, 400, 500, 600, 700, 800, 900, 1000, 1200, 1400, 1600, 1800, 2000, or more than 2000 iterations. A final cutoff threshold value is then determined at step 312 to separate samples exhibiting a microsatellite instability status of MSI-H from other samples by averaging over the series of candidate cutoff threshold values. The final cutoff threshold value is then validated at step 314 against the remaining patient data in the test data set.

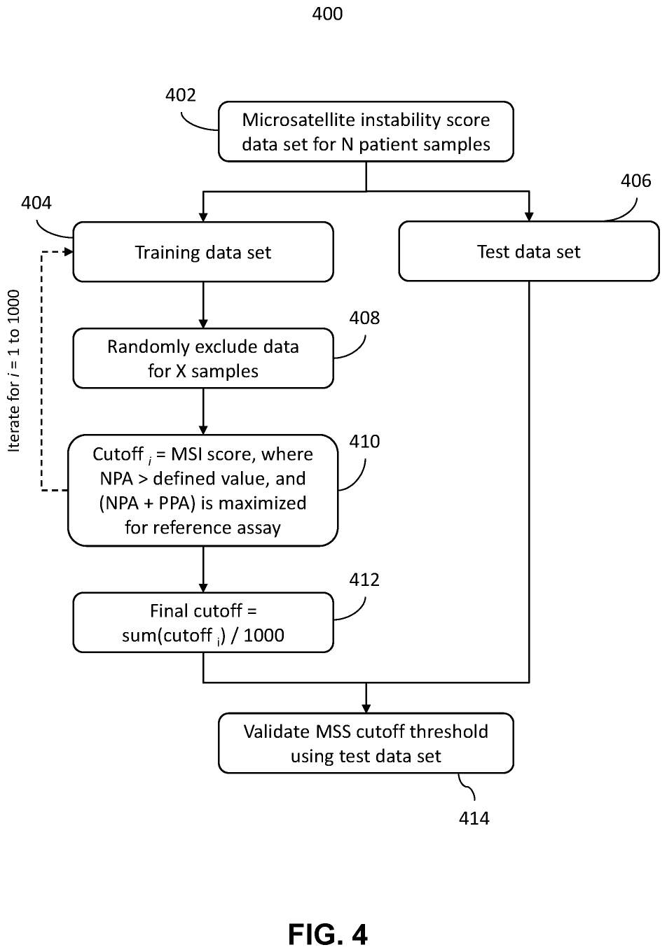

FIG. 4 provides a non-limiting example of a process 400 used to determine a threshold for distinguishing between microsatellite stable (MSS) samples and other samples based on a microsatellite instability (MSI) score. Microsatellite instability (MSI) score data for a plurality of patient samples, 402, is divided into a training data set, 404, and a test data set, 406, where the data split is stratified by MSI status as determined by a reference method and by cancer type(s) to ensure balanced sets of training and test data (e.g., so that the training and test data sets contain equivalent mixes of data for different cancer types and MSI status). In each iteration of the threshold determination process, at step 408, a subset of the training data is created by randomly excluding, e.g., 1%, 2%, 3%, 4%, 5%, 6%, 7%, 8%, 9%, 10%, or more than 10% of the samples, and, at step 410, a candidate cutoff threshold value is determined for the remaining data in the subset that maximizes the concordance of the training data subset with the results (e.g., the microsatellite—stable (MSS) results) obtained using a reference assay for microsatellite instability (e.g., the Promega Microsatellite Instability (MSI) Analysis assay, Promega Corporation, Madison, WI). For example, the candidate cutoff threshold value for the training data subset may be adjusted to maximize concordance with the results of the reference assay by requiring that negative percent agreement (NPA) be greater than a defined value (e.g., 85%, 86%, 87%, 88%, 89%, 90%, 91%, 92%, 93%, 94%, 95%, 96%, 97%, 98%, or 99%) while the sum of positive percent agreement (PPA) and negative percent agreement (NPA) be maximized. A series of candidate cutoff threshold values are determined by iterating through the steps of defining a training data subset by randomly excluding some of the training data at step 408 and determining a cutoff threshold value that maximizes concordance with the reference assay at step 410, where the steps are repeated for, e.g., at least 100, 200, 300, 400, 500, 600, 700, 800, 900, 1000, 1200, 1400, 1600, 1800, 2000, or more than 2000 iterations. A final cutoff threshold value is then determined at step 412 to separate samples exhibiting a microsatellite instability status of MSS from other samples by averaging over the series of candidate cutoff threshold values. The final cutoff threshold value is then validated at step 414 against the remaining patient data in the test data set.

In some instances, the plurality of microsatellite loci selected for evaluation according to the methods disclosed herein may range from about 100 microsatellite loci to about 3000 microsatellite loci. In some instances, the plurality of microsatellite loci selected for evaluation according to the methods disclosed herein may comprise at least 100, at least 200, at least 300, at least 400, at least 500, at least 600, at least 700, at least 800, at least 900, at least 1000, at least 1200, at least 1400, at least 1600, at least 1800, at least 2000, at least 2200, at least 2400, at least 2600, at least 2800, or at least 3000 microsatellite loci. In some instances, the plurality of microsatellite loci selected for evaluation according to the methods disclosed herein may comprise at most 3000, at most 2800, at most 2600, at most 2400, at most 2200, at most 2000, at most 1800, at most 1600, at most 1400, at most 1200, at most 1000, at most 900, at most 800, at most 700, at most 600, at most 500, at most 400, at most 300, at most 200, or at most 100 microsatellite loci. Any of the lower and upper values described in this paragraph may be combined to form a range included within the present disclosure, for example, in some instances the plurality of microsatellite loci selected for evaluation according to the methods disclosed herein may range from about 400 to about 2800 microsatellite loci. Those of skill in the art will recognize that the plurality of microsatellite loci selected for evaluation according to the methods disclosed herein may have any value within this range, e.g., about 2,382 microsatellite loci.

In some instances, the microsatellite loci to be evaluated for the presence of variants and used for the determination of MSI status may comprise mononucleotide, dinucleotide, trinucleotide repeat sequences (or motifs), or any combination thereof. In some instances, the microsatellite loci to be evaluated for the presence of variants and used for determination of MSI status may comprise repeat sequences (or motifs) of 1, 2, 3, 4, 5, 6, 7, 8, 9, 10, 11, 12, 13, 14, 15, or more than 15 nucleotides (or base pairs) in length.

In some instances, the microsatellite loci to be evaluated for the presence of variants and used for determination of MSI status may comprise 2, 3, 4, 5, 6, 7, 8, 9, 10, 15, 20, 25, 30, 35, 40, 45, 50, or more than 50 repeats of a given repeat sequence or motif.

In some instances, the overall length of microsatellite alleles to be evaluated for the presence of variants and used to determine MSI status may be at least 4 base pairs, at least 6, at least 8, at least 10, at least 12, at least 14, at least 16, at least 18, at least 20, at least 30, at least 40, at least 50, at least 60, at least 70, at least 80, at least 90, at least 100, at least 150, at least 200, at least 250, at least 300, at least 350, at least 400, at least 450, or at least 500 base pairs.

In some instances, the change in overall length of a detected variant microsatellite allele may be ±1, ±2, ±3, ±4, ±5, ±6, ±7, ±8, ±9, ±10, or more than ±10 nucleotides (or base pairs).

In some instances, as noted above, exclusion criteria may be applied to the input microsatellite allele sequence data to, e.g., exclude those microsatellite loci for which sequencing coverage fails to meet a defined minimum threshold from further analysis. In some instances, the minimum sequencing coverage required may be at least 100×, at least 125×, at least 150×, at least 175×, at least 200×, at least 225×, at least 250×, at least 275×, at least 300×, at least 400×, at least 500×, at least 600×, at least 700×, at least 800×, at least 900×, at least 1000×, at least 1200×, at least 1400×, at least 1600×, at least 1800×, or at least 2000×. In some instances, the minimum sequencing coverage required may be any value within the range of values described in this paragraphs, for example, at least 232×. In some instances, the minimum sequencing coverage required may be locus-dependent, i.e., different minimum sequence coverages may be required for different microsatellite loci.

In some instances, as noted above, a set of exclusion criteria may be applied to exclude microsatellite loci from further analysis that exhibit allele sequences that, for example, fail to meet a minimum allele frequency requirement (e.g., a minimum allele frequency of 2%, 3%, 4%, 5%, 6%, 7%, 8%, 9%, or 10%), that correspond to known germline alleles, that correspond to known sequencing errors, that differ in allele frequency from the mean allele frequency for that locus in a reference database by less than a specified amount (e.g., by less than one standard deviation, less than two standard deviations, less than three standard deviations, or less than four standard deviations), and the like. In some instances, these exclusion criteria may be locus-dependent, i.e., different exclusion criteria may be applied for different microsatellite loci or for different groups of microsatellite loci. For example, as noted, in comparing observed allele frequencies to the mean allele listed in a reference database, a different exclusion criteria may be applied in the case of short DNA repeat motifs (e.g., excluding alleles exhibiting an allele frequency of less than the expected mean allele frequency+two standard deviations for DNA repeat motifs of fewer than 10 bases) than that applied for longer DNA repeat motifs (e.g., excluding alleles exhibiting an allele frequency of less than the expected mean allele frequency+three standard deviations for DNA repeat motifs of 10 or more bases), due to the higher noise in allele frequency expected for longer DNA repeat motifs.

In some instances, the number of patient samples used to generate the MSI score data required for setting the first cutoff threshold (i.e., an MSI score threshold used to distinguish between MSI-H samples and other samples) and/or the second cutoff threshold (i.e., an MSI score threshold used to distinguish between MSS samples and other samples) may be at least 100, at least 250, at least 500, at least 750, at least 1000, at least 1250, at least 1500, at least 1750, at least 2000, at least 2500, at least 3000, at least 3500, at least 4000, at least 4500, at least 5000, or more than 5000 patient samples. In some instances, the number of patient samples used to generate the MSI score data required for setting the first cutoff threshold and/or the second cutoff threshold may have any value within the range of values described in this paragraph. e.g., at least 1,157 patient samples.

In some instances, the number of iterations of reference assay concordance optimization used to set the first and/or second cutoff thresholds (as illustrated in the non-limiting examples provided in FIG. 3 and FIG. 4) may be at least 100, at least 200, at least 300, at least 400, at least 500, at least 600, at least 700, at least 800, at least 900, at least 1000, at least 1200, at least 1400, at least 1600, at least 1800, or at least 2000. In some instances, the number of iterations of reference assay concordance optimization used to set the first and/or second thresholds may have any value within the range of values described in this paragraph, e.g., 1,225 iterations.

In some instances, the first and/or second cutoff thresholds may be set to maximize concordance with the results of a reference assay by requiring for example, that positive percent agreement (PPA), negative percent agreement (NPA), the sum of positive percent agreement and negative percent agreement (PPA+NPA), or a function of PPA and/or NPA be maximized. In some instances, positive percent agreement (PPA), negative percent agreement (NPA), the sum of positive percent agreement and negative percent agreement (PPA+NPA), or a function of PPA and/or NPA may be maximized while also requiring that PPA and/or NPA be greater than 0.85, greater than 0.90, greater than 0.91, greater than 0.92, greater than 0.93, greater than 0.94, greater than 0.95, greater than 0.96, greater than 0.97, greater than 0.98, greater than 0.99, or greater than 0.995.

As noted above, in some instances the reference method used to set the first and/or second cutoff thresholds be the Promega Microsatellite Instability (MSI) Analysis assay (Promega Corporation, Madison. WI). Other examples of reference methods that may be used include, but are not limited to, the TrueMark MSI Assay from ThermoFisher Scientific (Waltham, MA), the Bio-Rad ddPCR MSI Assay (Bio-Rad Laboratories, Hercules, CA), and the Idylla™ MSI Assay (Biocartis US, Inc., Jersey City, NJ).

In some instances, the numerical value of the first cutoff threshold (i.e., the MSI score threshold used to distinguish between MSI-H samples and other samples) may range from about 0.0100 to about 0.0150. In some instances, the numerical value of the first cutoff threshold may be at least 0.0100, at least 0.0105, at least 0.0110, at least 0.0115, at least 0.0120, at least 0.0125, at least 0.0130, at least 0.0135, at least 0.0140, at least 0.0145, or at least 0.0150. In some instances, the numerical value of the first cutoff threshold may be at most 0.0150, at most 0.0145, at most 0.0140, at most 0.0135, at most 0.0130, at most 0.0125, at most 0.0120, at most 0.0115, at most 0.0110, at most 0.0105, at most 0.0100. Any of the lower and upper values described in this paragraph may be combined to form a range included within the present disclosure, for example, in some instances the numerical value of the first cutoff threshold may range from about 0.0115 to about 0.0135. Those of skill in the art will recognize that the numerical value of the first cutoff threshold may have any value within this range, e.g., about 0.0124.

In some instances, the numerical value of the second cutoff threshold (i.e., the MSI score threshold used to distinguish between MSS samples and other samples) may range from about 0.0001 to about 0.0060. In some instances, the numerical value of the first cutoff threshold may be at least 0.0001, at least 0.0005, at least 0.0010, at least 0.0015, at least 0.0020, at least 0.0025, at least 0.0030, at least 0.0035, at least 0.0040, at least 0.0045, at least 0.0050, at least 0.0055, or at least 0.0060. In some instances, the numerical value of the first cutoff threshold may be at most 0.0060, at most 0.0055, at most 0.0050, at most 0.0045, at most 0.0040, at most 0.0035, at most 0.0030, at most 0.0025, at most 0.0020, at most 0.0015, at most 0.0010, at most 0.0005, or at most 0.0001. Any of the lower and upper values described in this paragraph may be combined to form a range included within the present disclosure, for example, in some instances the numerical value of the second cutoff threshold may range from about 0.0005 to about 0.0045. Those of skill in the art will recognize that the numerical value of the second cutoff threshold may have any value within this range, e.g., about 0.0041.

### Methods of Use:

As noted above, in some instances the disclosed methods for determination of the MSI status of a sample derived from a subject (e.g., a patient) may be used alone or in combination with other diagnostic tests for the detection of cancer, diagnosis of cancer, providing a disease prognosis, selecting a cancer therapeutic, and/or monitoring tumor progression.

For example, in some instances a determination of MSI status may serve as a biomarker for detection and/or diagnosis of a cancer. Although particularly prevalent in colorectal cancer, determination of MST at any microsatellite locus represents a potential clonal marker for the detection of cancer (Boland, et al. (1998), ibid.). For example, studies have indicated that a determination of MSI status can served as a biomarker for the detection and monitoring of bladder cancer. In combination with loss of heterozygosity (LOH) studies, the majority of bladder cancers were identified by microsatellite instability analysis of urine samples—in some cases before the direct visualization of microscopic tumors by cystoscopy (Boland, et al. (1998), ibid.). MSI status may also be a useful biomarker for the detection of other tumor types by analysis of bodily fluids such as the serum or plasma of cancer patients. Examples of cancers or tumor types for which MSI status may serve as a biomarker for detection and/or diagnosis include, but are not limited to, bladder cancer, brain cancer, breast cancer, colorectal cancer, gastrointestinal cancer, kidney cancer, liver cancer, lung cancer, ovarian cancer, pancreatic cancer, prostate cancer, rectal cancer, skin cancer, and uterine cancer, leukemia, lymphomas, and endometrial cancer.

In some instances, a determination of MSI status may serve as a biomarker for disease prognosis, e.g., a prognosis for a cancer. For example, MSI status may serve as a significant biomarker for prognosis and adjuvant therapy of colorectal cancer that is dependent on the stage of the cancer. Studies have indicated that patients with stage I and stage II MSI-H colorectal cancer have a good prognosis, a high 5-year survival rate, and a low recurrence rate, while patients with stage III MSI-H colorectal cancer have the opposite prognosis. Examples of cancers or tumor types for which MSI status may serve as a biomarker for disease prognosis include, but are not limited to, bladder cancer, brain cancer, breast cancer, colorectal cancer, gastrointestinal cancer, kidney cancer, liver cancer, lung cancer, ovarian cancer, pancreatic cancer, prostate cancer, rectal cancer, skin cancer, uterine cancer, leukemia, lymphomas, and endometrial cancer.

In some instances, a determination of MSI status may serve as a biomarker for selection, initiation, adjustment of dosage, and/or termination of a cancer therapy (e.g., a small molecule therapeutic drug, an immunotherapy, a chemotherapy, or a targeted therapy). For example, microsatellite instability status is now being used as a biomarker to guide immunotherapy treatment for men with advanced prostate cancer. The U.S. Food and Drug Administration (FDA) also recently granted approval of an immunotherapy-based anti-PD-1 cancer treatment (pembrolizumab) for patients whose cancers exhibit MSI or deficient DNA mismatch repair (dMMR). MSI-H status or MMR deficiency also appears to account for some of the resistance to cisplatin treatment in patients with ovarian cancer. Table 1 provides a non-limiting summary of MSI-H related diseases for which MSI status may be a useful biomarker for prognosis and treatment options.

TABLE 1
Disease prognosis and treatment options for MSI-H samples.

| Disease | Prognosis | Treatment Options |
|---|---|---|
| Lynch | MSI-H (dMMR) status is | Aspirin/sulinda may play a |
| Syndrome | indicative of a good | preventive role in reducing |
| | prognosis | the risk of Lynch |
| | | syndrome-related cancer, |
| | | especially in patients with |
| | | hMSH2 and hMLH1 gene |
| | | alterations |
| Colorectal | MSI-H status in stage I | 5-FU not recommended as |
| cancer | and stage II CRC is | a chemotherapeutic agent |
| (CRC) | indicative of a good | for stage I or stage II |
| | prognosis; | CRC patients; |
| | MSI-H status in stage | 5-FU may be used as a |
| | III CRC is indicative | chemotherapeutic agent |
| | of a poor prognosis | for stage III to IV CRC |
| | | patients; |
| | | An anti-PD-1/PDL-1 |
| | | treatment may be used for |
| | | CRC patients at different |
| | | stages |
| Gastric | MSI-H status in | Surgical treatment; avoid |
| cancer | resectable primary gastric | adjuvant chemotherapy in |
| (GC) | cancer is indicative of | MSI-H GC |
| | a good prognosis | |
| Breast | MSI-H status is | Olaparib used in |
| cancer | indicative of a poor | combination with other |
| | prognosis | drugs, such as platinum, |
| | | can strengthen the |
| | | combined effect |
| Prostate | MSI-H (dMMR) status in | Anti-PD-1/PDL-1 treatment |
| cancer | patients with prostate | |
| | cancer is indicative | |
| | of a good prognosis | |
| Leukemia | MSI-H (dMMR) status in | Anti-PD-1/PDL-1 treatment |
| | patients with leukemia | |
| | is indicative of a good | |
| | prognosis | |
| Bladder | MSI-H (dMMR) status in | Anti-PD-1/PDL-1 treatment |
| cancer | patients with bladder | |
| | cancer is indicative of | |
| | a good prognosis | |
| Ovarian | MSI-H status in patients | Anti-PD-1/PD-L1 drugs |
| cancer | with clear-cell ovarian | |
| | carcinomas | |
| | (CCOCs) is indicative | |
| | of favorable | |
| | response to | |
| | immunotherapy | |
| Endometrial | MSI-H status for middle | Use anti-PD-1/ PD-L1 |
| cancer | and late stage | drugs and chemotherapeutic |
| | endometrial cancer is | drugs such as |
| | associated with | temozolomide and cisplatin |
| | a poor prognosis | |
| Pancreatic | MSI-H (dMMR) status in | Anti-PD-1/PD-L1 drugs |
| ductal | patients with PDAC is | |
| adenocarcinoma | indicative of a good | |
| (PDAC) | prognosis | |
| Follicular | MSI-H status in patients | Anti-PD-1/PD-L1 drugs |
| thyroid | with FTC is indicative | |
| cancer | of a prolonged survival | |
| (FTC) | time | |

In some instances, a determination of MSI status may serve as a biomarker for monitoring tumor progression and/or response to a therapeutic treatment of disease (e.g., cancer). For example, pilot studies have indicated the potential use of MSI status for detection and monitoring of bladder cancer (Boland, et al. (1998), ibid.). MSI status may be determined and compared for samples drawn from a subject (e.g., a patient) having cancer at two or more different time points, where a change in MSI status (e.g., a change from a status of MSI-H to a status of MSI-E or MSS) is indicative of the subject's response to a treatment. In some instances, a treatment or therapy for disease (e.g., a treatment or therapy for cancer) may be adjusted, replaced, or terminated based on a detected change in MSI status. In some instances, a first time point may correspond to a time before which the treatment was administered to the subject, and second, third, fourth, etc., time points may correspond to times following the treatment (or following initiation of the treatment) of the subject. In some instances, a series of 2, 3, 4, 5, 6, 7, 8, 9, 10, 20, 30, 40, 50, or more than 50 samples may be collected at different times from the subject for analysis of MSI status using the methods disclosed herein. In some instances, samples may be collected from the subject at periodic, variable, or random time intervals. For example, in some instances, samples may be collected daily, weekly, monthly, semi-annually, or annually, or on any other time interval that is appropriate for evaluating the effectiveness of a treatment or therapy.

In some instances, any of the methods disclosed herein may further comprise obtaining a sample, e.g., a patient sample described herein. The sample can be acquired directly or indirectly. In some instances, the sample is acquired, e.g., by isolation or purification, from a sample that comprises cell-free DNA (cfDNA). In some instances, the sample is acquired, e.g., by isolation or purification, from a sample that comprises circulating tumor DNA (ctDNA). In some instances, the sample is acquired, e.g., by isolation or purification, from a sample that comprises both malignant cells and non-malignant cells (e.g., tumor-infiltrating lymphocytes). In some instances, the sample is acquired, e.g., by isolation or purification, from a sample that comprises circulating tumor cells (CTCs). In some embodiments, the sample is obtained by surgical resection or tissue biopsy (e.g., a solid tissue biopsy).

In some instances, any of the methods disclosed herein may comprise preparing a sequencing library. A sequencing library can be prepared from a patient sample using known methods. The nucleic acid molecules may be purified or isolated from the patient sample. In some instances, the isolated nucleic acids are fragmented or sheared using a known method. For example, nucleic acid molecules may be fragmented by physical shearing methods (e.g., sonication), enzymatic cleavage methods, chemical cleavage methods, and other methods well known to those skilled in the art. In some instances, the nucleic acid may be ligated to an adapter sequence for sequencing. In some instances, the adapter may comprise an amplification primer and/or sequencing adapter. In some instances, nucleic acid molecules purified or isolated from the patient sample, or the sequencing library prepared therefrom, may be amplified, e.g., using a polymerase chain reaction (PCR) or isothermal amplification method known to those of skill in the art. Examples of non-PCR based amplification methods that may be used include, but are not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. Examples of isothermal amplification methods that may be used include, but are not limited to, loop-mediated isothermal amplification (LAMP), helicase-dependent amplification (HDA), rolling circle amplification (RCA), multiple displacement amplification (MDA), whole genome amplification (WGA), and recombinase polymerase amplification (RPA).

In some instances, the nucleic acid molecules extracted from the patient sample and used to prepare a sequencing library (or a selected (e.g., captured) subset thereof) are sequenced to generate a patient genomic sequence (or portion thereof). Sequencing methods are well known in the art, and may be performed using multiplexed (e.g., next-generation) or single molecule sequencing. The patient genomic sequence determined by sequencing need not be the full genome of the patient. For example, in some instances, targeted sequencing methods (e.g., using specific probes (or bait) molecules for hybridization-based capture) are used to sequence portions of the patient's genome (i.e., less than the full genome). See, for example, U.S. Pat. No. 9,340,830 B2. Targeted sequencing may be used to target, for example, one or more exon regions, one or more intron regions, one or more intragenic regions, one or more 3□-UTRs (untranslated regions), and/or one or more 5′-UTRs.

In some instances, any of the method disclosed herein may further comprise displaying a user interface comprising the MSI status via an online portal, e.g., to a healthcare provider. In some instances, any of the methods disclosed herein may further comprise displaying a user interface comprising the MSI status via a mobile device. In some instances, the user interface may comprise an MSI score data structure field. In some instances, any of the methods disclosed herein may further comprise generating a report of the determined MSI status, transmitting the report of the MSI status to a healthcare provider through a computer network, and/or transmitting the report of MSI status to a healthcare provider over the Internet or via a peer-to-peer connection.

In some instances, the disclosed methods may comprise determining, identifying, and/or applying the MSI status of a sample derived from a subject (e.g., a patient) as a diagnostic value associated with the sample. In some instances, the MSI status of the sample is used in making suggested treatment decisions for the subject. For example, the MSI status of the sample may be used in suggesting an anti-cancer agent (or anti-cancer therapy, e.g., any drug that is effective in the treatment of malignant, or cancerous, disease, including, but not limited to alkylating agents, antimetabolites, natural products, and hormones), chemotherapy, radiation therapy, immunotherapy, surgery, or a therapy configured to target a defect in a DNA mismatch repair (MMR) pathway. In some instances, the MSI status of the sample may be used in applying or administering a treatment to the subject.

In some instances, the disclosed methods may further comprise using the determined MSI status in generating a genomic profile for the subject. As noted elsewhere herein, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors. Inclusion of the disclosed methods for determining microsatellite instability as part of a genomic profiling process can improve the validity of, e.g., disease detection calls, made on the basis of the genomic profiling by, for example, independently confirming the presence of an impaired DNA mismatch repair (MMR) mechanism in a given patient sample.

### Samples:

The disclosed methods and systems may be used to determine microsatellite instability status in any of a variety of samples (also referred to herein as specimens) comprising nucleic acids (e.g., DNA or RNA) that are collected from a subject (e.g., a patient). Examples include, but are not limited to, a tissue sample, a biopsy sample, a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavages or bronchoalveolar lavages), etc. In some instances, the sample is acquired from a liquid biopsy, which can be from, e.g., whole blood, blood plasma, blood serum, urine, saliva, or cerebrospinal fluid.

In some instances, the sample comprises one or more premalignant or malignant cells. Premalignant, as used herein, refers to a cell or tissue that is not yet malignant but is poised to become malignant. In certain instances, the sample is acquired from a solid tumor, a soft tissue tumor, or a metastatic lesion. In certain instances, the sample is acquired from a hematologic malignancy or pre-malignancy. In other instances, the sample comprises a tissue or cells from a surgical margin. In certain instances, the sample comprises tumor-infiltrating lymphocytes. In some instances, the sample comprises one or more non-malignant cells. In some instances, the sample is, or is part of, a primary tumor or a metastasis (e.g., a metastasis biopsy sample). In some instances, the sample is obtained from a site (e.g., a tumor site) with the highest percentage of tumor (e.g., tumor cells) as compared to adjacent sites (e.g., sites adjacent to the tumor). In some instances, the sample is obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as microsatellite instability as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis and circulate in the blood stream and/or accumulate in other bodily fluids. Circulating tumor DNA (ctDNA) is comprises of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, e.g., for the analysis of microsatellite loci located in coding regions, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), messenger RNA (mRNA) or fragments thereof, the poly(A)—tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)—containing oligonucleotides. Methods for depletion, poly(A) enrichment, and cDNA synthesis are well known to those of skill in the art.

In some instances, the sample may comprise a tumor content, e.g., comprising tumor nuclei. In some instances, the sample may comprise a tumor content of at least 5-50%, 10-40%, 15-25%, or 20-30% tumor nuclei. In some instances, the sample may comprise a tumor content of at least 5%, at least 10%, at least 20%, at least 30%, at least 40%, or at least 50% tumor nuclei. In some instances, the percent tumor nuclei is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice or more than twice the DNA content of other, e.g., non-hepatocyte, somatic nuclei. In some instances, the sensitivity of detection of an genetic alteration, e.g., as evidenced by a determination of microsatellite instability as described herein, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., a cell, protein, carbohydrate, or lipid, e.g., from the tumor or normal tissue.

### Subjects:

In some instances, the sample is obtained (e.g., collected) from a subject (e.g., patient) with a condition or disease. e.g., a hyperproliferative disease or a non-cancer indication. In some instances, the hyperproliferative disease is a cancer. In some instances, the cancer is a solid tumor or a metastatic form thereof. In some instances, the cancer is a hematological cancer, e.g. a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with a cancer therapy. In some instances, the subject is in need of being monitored for relapse of cancer. In some instances, the subject has been, or is being treated, for cancer. In some instances, the subject has not been treated with a cancer therapy.

In some instances, the subject (e.g., a patient) has been previously treated with one or more targeted therapies. In some instances, for a patient who has been previously treated with a targeted therapy, a post-targeted therapy sample, e.g., specimen, is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a recurrence (e.g., a disease recurrence post-therapy).

### Cancers:

In some instances, the sample is acquired from a subject having a cancer. Exemplary cancers include, but are not limited to, B cell cancer (e.g. multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithclial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLIJSLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sdzary syndrome, Waldenström macroglobulinemia), chronic myeloproliferative neoplasm, Langerhans cell histiocytosis, multiple mycloma/plasma cell neoplasm, myclodysplastic syndrome, or myelodysplastic/myeloproliferative neoplasm.

### Nucleic Acid Extraction and Processing:

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell, 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation. Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted. (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42: Masuda, et al., (1999) Nucleic Acids Res. 27(22):4436-4443; Specht, et al., (2001) Am JPathol. 158(2):419-429; the Ambion RecoverAll® Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp_DNA FFPE Tissue Handbook (Qiagen, Cat. No. 37625, October 2007). For example, the RccoverAll™ Total Nucleic Acid Isolation Kit uses xylene at elevated temperatures to solubilize paraffin-embedded samples and a glass-fiber filter to capture nucleic acids. The Maxwellt 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise acquiring a yield value for the nucleic acid extracted from the sample and comparing the acquired value to a reference value. For example, if the acquired value is less than the reference value, the nucleic acids may be amplified prior to proceeding with library construction. In some instances, the disclosed methods may further comprise acquiring a value for the size (or average size) of nucleic acid fragments in the sample, and comparing the acquired value to a reference value, e.g., a size (or average size) of at least 100, 200, 300, 400, 500, 600, 700, 800, 900, or 1000 base pairs (bps). A parameter described herein can be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation:

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., adapters comprising amplification primer sequences (amplification primer binding sites, e.g., Illumina Rd1 SP and Rd2 SP sequences), flow cell or substrate sequencing adapters (e.g., Illumina P5 and P7 sequences for library sequence binding and generation of clusters on flow cell or substrate surfaces),sample barcode or sample index sequences for pooling/multiplexing of samples, and/or unique molecular identifier sequences), size-selected (e.g., by preparative gel electrophoresis), and/or amplified (e.g., using PCR). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and illumina's genomic DNA sample preparation kit.

In some instances, the resulting nucleic acid library can contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single individual. In some instances, a library can comprise nucleic acid molecules derived from more than one subject (e.g., 2, 3, 4, 5, 6, 7, 8, 9, 10, 20, 30 or more subjects), e.g., two or more libraries from different subjects can be combined to form a library having nucleic acid molecules from more than one subject. In some instances, the subject is a human having, or at risk of having, a cancer or tumor.

### Targeting Microsatelite Loci for Analysis:

The methods described herein can be used in combination with, or as part of, a method for evaluating a plurality or set of subject intervals (e.g., target sequences), e.g., from a set of genomic loci (e.g., genes, microsatellite loci, etc.), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of genomic loci evaluated by the disclosed methods comprises a plurality of microsatellite loci encoding alleles, which in variant (mutant) form, are associated with a cancer, e.g., a cancer described herein. In some instances, the detection of variant alleles at a plurality of microsatellite loci provides phenotypic evidence that there is a deficient DNA mismatch repair mechanism in the tissue. e.g., the cancerous tissue, from which the sample being analyzed was collected.

In some instances, the set of microsatellite loci comprises at least about 50 or more, about 100 or more, about 150 or more, about 200 or more, about 250 or more, about 300 or more, about 350 or more, about 400 or more, about 450 or more, about 500 or more, about 550 or more, about 600 or more, about 650 or more, about 700 or more, about 750 or more, about 800 or more, about 850 or more, about 900 or more, about 950 or more, about 1,000 or more, about 1,200 or more, about 1,400 or more, about 1,600 or more, about 1,800 or more, about 2,000 or more, about 2,500 or more, about 3,000 or more, about 3,500 or more, about 4,000 or more, about 4,500 or more, or about 5,000 or more microsatellite loci.

In some instances, the selected microsatellite loci (also referred to herein as the “target” microsatellite loci) may include subject intervals (or target nucleic acid sequences) comprising non-coding regions, coding regions, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents:

The methods described herein may comprise contacting a nucleic acid library with a plurality of target capture reagents in order to select and capture a plurality of specific target sequences (e.g., microsatellite sequences) for analysis. In some instances, a target capture reagent (i.e., a molecule which can bind to and thereby allow capture of a target molecule) is used to select the subject intervals to be analyzed. For example, a target capture reagent can be a bait, e.g., a nucleic acid molecule (e.g., a DNA molecule or RNA molecule), which can hybridize to (e.g., is complementary to) a target molecule, and thereby allows capture of the target nucleic acid. In some instances, the target capture reagent, e.g., bait, is a capture oligonucleotide. In some instances, the target nucleic acid is a genomic DNA molecule, an RNA molecule, or a cDNA molecule derived from an RNA molecule, a microsatellite DNA sequence, and the like.

In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target microsatellite locus. In some instances, a target capture reagent may hybridize to a specific group of target loci, e.g., a specific group of microsatellite loci. In some instances, a target capture reagent may hybridize to a specific locus that comprises more than one microsatellite locus. In some instances, a plurality of target capture reagents comprising a mix of target-specific and/or group-specific target capture reagents may be used.

In some instances, the number of target capture reagents (e.g., baits) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25.000, or greater than 50,000.

In some instances, the overall length of the target capture reagent sequence can be between about 25 nucleotides and 1000 nucleotides. In one embodiment, the target capture reagent length is between about 25 and 400, 100 and 300 nucleotides, 110 and 200 nucleotides, or 120 and 170 nucleotides, in length. In addition to those mentioned above, intermediate oligonucleotide lengths of about 30, 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 30, 40, 50, 60, 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a nucleic acid molecule-specific or microsatellite locus-specific complementary sequence), (ii) an adapter, primer, barcode, and/or unique molecular identifier sequence, and (iii) universal tails on one or both ends. As used herein, the term “target capture reagent sequence” can refer to the target-specific target capture sequence or the entire oligonucleotide including the target-specific target capture sequence and other nucleotides of the oligonucleotide.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent sequences may be designed to capture the forward strand sequence, the reverse complement strand sequence, or both the forward and reverse strand sequences for the same target nucleic acid molecule sequence.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

The disclosed methods may comprise the use of one, two, three, four, five, or more than five pluralities of target capture reagents, where each plurality is designed to capture target sequences in a different target category. In some instances, any combination of two, three, four, five, or more than five pluralities of target capture reagents can be used, for example, a combination of first and second pluralities of target capture reagents; first and third pluralities of target capture reagents; first and fourth pluralities of target capture reagents; first and fifth pluralities of target capture reagents; second and third pluralities of target capture reagents; second and fourth pluralities of target capture reagents; second and fifth pluralities of target capture reagents; third and fourth pluralities of target capture reagents; third and fifth pluralities of target capture reagents; fourth and fifth pluralities of target capture reagents; first, second and third pluralities of target capture reagents: first, second and fourth pluralities of target capture reagents; first, second and fifth pluralities of target capture reagents: first, second, third, and fourth pluralities of target capture reagents; first, second, third, fourth and fifth pluralities of target capture reagents, and so on.

In some instances, each of the first, second, third, fourth, and fifth, etc., pluralities of target capture reagents has a unique recovery efficiency. In some instances, at least two or three pluralities of target capture reagents have recovery efficiency values that differ.

In some instances, the value for recovery efficiency is modified by one or more of: differential representation of different target capture reagents, differential overlap of target capture reagent subsets, differential target capture reagent parameters, mixing of different target capture reagents, and/or using different types of target capture reagents. For example, a variation in recovery efficiency (e.g., relative sequence coverage of each target capture reagent/target category) can be adjusted, e.g., within a plurality of target capture reagents and/or among different pluralities of target capture reagents, by altering one or more of:
(i) Differential representation of different target capture reagents—The target capture reagent design to capture a given target (e.g., a target nucleic acid molecule) can be included in more/fewer number of copies to enhance/reduce relative target sequencing depths:
(ii) Differential overlap of target capture reagent subsets—The target capture reagent design to capture a given target (e.g., a target nucleic acid molecule) can include a longer or shorter overlap between neighboring target capture reagents to enhance/reduce relative target sequencing depths:
(iii) Differential target capture reagent parameters—The target capture reagent design to capture a given target (e.g., a target nucleic acid molecule) can include sequence modifications/shorter length to reduce capture efficiency and lower the relative target sequencing depths:
(iv) Mixing of different target capture reagents—Target capture reagents that are designed to capture different target sets can be mixed at different molar ratios to enhance/reduce relative target sequencing depths:
(v) Using different types of oligonucleotide target capture reagents—In certain instances, the target capture reagent can include:
(a) one or more chemically (e.g., non-enzymatically) synthesized (e.g., individually synthesized) target capture reagents.
(b) one or more target capture reagents synthesized in an array,
(c) one or more enzymatically prepared, e.g., in vitro transcribed, target capture reagents: (d) any combination of (a), (b) and/or (c).
(e) one or more DNA oligonucleotides (e.g., a naturally or non-naturally occurring DNA oligonucleotide),
(f) one or more RNA oligonucleotides (e.g., a naturally or non-naturally occurring RNA oligonucleotide),
(g) a combination of (e) and (f), or
(h) a combination of any of the above.

The different oligonucleotide combinations can be mixed at different ratios, e.g., a ratio chosen from 1:1, 1:2, 1:3, 1:4, 1:5, 1:10, 1:20, 1:50: 1:100, 1:1000, or the like. In one embodiment, the ratio of chemically-synthesized target capture reagent to array-generated target capture reagent is chosen from 1:5, 1:10, or 1:20.

Typically. DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

Target capture reagents comprising DNA or RNA oligonucleotides can include naturally—or non-naturally-occurring nucleotides. In some instances, the target capture reagents include one or more non-naturally-occurring nucleotides to, e.g., increase melting temperature. Exemplary non-naturally occurring oligonucleotides include modified DNA or RNA nucleotides. Exemplary modified nucleotides (e.g., modified RNA or DNA nucleotides) include, but are not limited to, locked nucleic acids (LNAs), wherein the ribose moiety of an LNA nucleotide is modified with an extra bridge connecting the 2′ oxygen and 4′ carbon; peptide nucleic acids (PNAs), e.g., a PNA composed of repeating N-(2-aminoethyl)-glycine units linked by peptide bonds; a DNA or RNA oligonucleotide modified to capture low GC regions; a bicyclic nucleic acid (BNA); a cross-linked oligonucleotide; a modified 5-methyl deoxycytidine; and 2,6-diaminopurine. Other modified DNA and RNA nucleotides are known in the art.

In some instances, a substantially uniform or homogeneous coverage of a target sequence (e.g., a target nucleic acid molecule) is obtained. For example, within each target capture reagent/target category, uniformity of coverage can be optimized by modifying target capture reagent parameters, for example, by one or more of:
(i) Increasing/decreasing target capture reagent representation or overlap can be used to enhance/reduce coverage of targets (e.g., target nucleic acid molecules), which are under/over-covered relative to other targets in the same category;
(ii) For low coverage, hard to capture target sequences (e.g., high GC content sequences), expand the region being targeted with the target capture reagents to cover, e.g., adjacent sequences (e.g., less GC-rich adjacent sequences);
(iii) Modifying a target capture reagent sequence can be used to reduce secondary structure of the target capture reagent and enhance its recovery efficiency;
(iv) Modifying a target capture reagent length can be used to equalize melting hybridization kinetics of different target capture reagents within the same category. Target capture reagent length can be modified directly (by producing target capture reagents with varying lengths) or indirectly (by producing target capture reagents of consistent length, and replacing the target capture reagent ends with arbitrary sequence):
(v) Modifying target capture reagents of different orientation for the same target region (i.e. forward and reverse strand) may have different binding efficiencies. The target capture reagent with either orientation providing optimal coverage for each target may be selected;
(vi) Modifying the amount of a binding entity, e.g., a capture tag (e.g. biotin), present on each target capture reagent may affect its binding efficiency. Increasing/decreasing the tag level of target capture reagents targeting a specific target may be used to enhance/reduce the relative target coverage;
(vii) Modifying the type of nucleotide used for different target capture reagents can be used to affect binding affinity to the target, and enhance/reduce the relative target coverage; or
(viii) Using modified oligonucleotide target capture reagents. e.g., having more stable base pairing can be used to equalize melting hybridization kinetics between areas of low or normal GC content relative to high GC content.

In some instances, the disclosed methods thus comprise providing a selected set of nucleic acid molecules (e.g., a library catch) captured from one or a plurality of nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects: contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by PCR). In other instances, the library catch is not amplified.

In some instances, the target capture reagents can be part of a kit which can optionally comprise instructions, standards, buffers or enzymes or other reagents.

### Hybridization Conditions:

As noted above, the methods disclosed herein may include the step of contacting the library (e.g., the nucleic acid library) with a plurality of target capture reagents to provide a selected library catch. The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou. D.T. et al. (2007) Nat. Methods 4(11):907-9, the contents of which are incorporated herein by reference in their entireties.

Hybridization methods that can be adapted for use in the methods herein are described in the art, e.g., as described in Intemational Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods:

The methods and systems disclosed herein can be used in combination with, or as part of, a method or system for sequencing nucleic acids (e.g., a next-generation sequencing system) to determine, e.g., microsatellite allele sequences at a plurality of microsatellite loci. “Next-generation sequencing” (or “NGS”) as used herein, refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. The disclosed methods and systems may be implemented using sequencing platforms such as the Roche 454, Illumina Solexa. ABI-SOLiD. ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. Optimized methods for sequencing a large number of target genomic loci in nucleic acids extracted from a sample are described in more detail in, e.g., Intemational Patent Application Publication No. WO 2020/236941, the entire content of which is incorporated herein by reference.

In certain instances, the disclosed methods comprise acquiring a library comprising a plurality of nucleic acid molecules (e.g., DNA or RNA molecules) derived from a sample from a subject. In certain instances, the methods further comprise contacting the library with target capture reagents (e.g., using solution-based hybridization or solid-phase hybridization of nucleic acid molecules to target capture probes and/or sequencing primers), thereby providing a targeted library or targeted subgroup thereof (e.g., a library catch) of subject intervals (e.g., subgenomic sequences or target sequences) that can be sequenced. In certain instances, the methods further comprise acquiring a sequencing read (e.g., using a next-generation sequencing method) for one or more subject intervals (target sequences) that correspond to one or more targeted genomic loci (e.g., microsatellite loci) that may comprise an alteration (e.g., a change in allele length). In certain instances, the disclosed methods further comprise aligning a read for the one or more subject intervals using an alignment method (e.g., an alignment method described herein). In certain instances, the methods further comprise identifying a nucleotide (or assigning a nucleotide value) for a given nucleotide position in a read for the one or more subject intervals, e.g., using a mutation calling method described herein. In certain instances, the methods may comprise re-sequencing all or a portion of the library catch.

In certain instances, the disclosed methods comprise one, two, three, four, five, or more of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules to provide a selected set of normal and/or tumor nucleic acid molecules, wherein said plurality of target capture reagents hybridize with the normal and/or tumor nucleic acid molecules, thereby providing a library catch; (c) separating the selected subset of the nucleic acid molecules (e.g., a library catch) from the hybridization mixture, e.g., by contacting the hybridization mixture with a binding entity that allows for separation of the target capture reagent/nucleic acid molecule hybrids from the hybridization mixture, (d) acquiring a read (e.g., a sequencing read) for one or more subject intervals (e.g., one or more target or subgenomic sequences) from said library catch that may comprise an alteration (e.g., a somatic alteration or variant allele) using, e.g., a next-generation sequencing method; (e) aligning said sequencing read using an alignment method (e.g., an alignment method described herein); and/or (f) assigning a nucleotide value for a nucleotide position of the subject interval (e.g., calling a mutation (e.g., using a Bayesian method or other method described herein)) from the sequencing read.

In some instances, acquiring a read for the one or more subject intervals comprises sequencing a subject interval for at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1.250, at least 1,500, at least 1,750, at least 2,000, at least 2,250, at least 2.500, at least 2,750, at least 3,000, at least 3,500, at least 4,000, at least 4,500, or at least 5,000 loci, e.g., microsatellite loci. In some instances, acquiring a read for the one or more subject intervals may comprise sequencing a subject interval for any number of loci, e.g., microsatellite loci, within the range described in this paragraph, e.g., at least 2,850 microsatellite loci.

In some instances, acquiring a read for the one or more subject intervals comprises sequencing a subject interval with a sequencing method that provides a read length (or average read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a read for the one or more subject intervals may comprise sequencing a subject interval with a sequencing method that provides a read length (or average read length) of any number of bases within the range described in this paragraph, e.g., a read length (or average read length) of 56 bases.

In some instances, acquiring a read for the one or more subject intervals comprises sequencing with at least 100× or more average depth. In some instances, acquiring a read for the subject interval comprises sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more average depth. In some instances, acquiring a read for the subject interval may comprise sequencing with an average depth having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the microsatellite loci sequenced. For example, in some instances acquiring a read for the subject interval comprises sequencing with an average sequencing depth of at least 125× for at least 99% of the microsatellite loci sequenced. As another example, in some instances acquiring a read for the subject interval comprises sequencing with an average sequencing depth of at least 4,100× for at least 95% of the microsatellite loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., microsatellite loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment:

Alignment is the process of matching a read with a location, e.g., a genomic location or locus. In some instances, NGS reads may be aligned to a known reference sequence (e.g., a wild-type sequence). In some instances. NGS reads may be assembled de novo. Methods of sequence alignment for NGS reads are described in, e.g., Trapnell, C. and Salzberg, S.L. Nature Biotech., 2009, 27:455-457. Examples of de novo sequence assemblies are described in, e.g., Warren R., et al., Bioinformatics, 2007, 23:500-501: Butler, J. et al., Genome Res., 2008, 18:810-820; and Zerbino, D. R. and Bimey, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

For the MSI status determination methods disclosed herein, sequencing reads should be mapped to the correct genomic locus, i.e., they are uniquely addressable to the specific genomic location. In some instances, an exhaustive realignment of sequencing reads (with careful consideration of flanking sequences) is used to make a determination of what allele is supported by each read, rather than the detailed alignment and mapping used to make mutation calls. In some instances, de novo assembly may be used instead of realignment to determine what allele is supported by each read.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions—deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing of a large number of diverse genetic events at a large number of diverse genomic loci (e.g., the methods for determining microsatellite instability described herein). In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007). “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequencing reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, sequencing reads from each of X unique subject intervals are aligned using from 1 to X unique alignment method(s), and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. In some instances, subject intervals from at least X genomic loci (e.g., at least X microsatellite loci) are aligned using from 1 to X unique alignment method(s), and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater.

In some instances, the methods disclosed herein further comprise selecting or using an alignment method for analyzing. e.g., aligning, a sequencing read, wherein said alignment method is a function of, is selected responsive to, or is optimized for, one or more of (i) tumor type, e.g., the tumor type in the sample; (ii) the location (e.g., a gene or microsatellite region) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., microsatellite loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C-T in melanoma): or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

In some instances, the disclosed methods may comprise the use of an alignment selector. An “alignment selector”, as used herein, refers to a parameter that allows or directs the selection of an alignment method, e.g., an alignment algorithm or parameter, that can optimize the sequencing performance for a subject interval. An alignment selector can be specific to, or selected as, a function of, e.g., one or more of the following:
1. The sequence context, e.g., a subject interval to be evaluated or a nucleotide position therein, that is associated with a propensity for misalignment of reads. For example, the existence of a sequence element in or near the subject interval to be evaluated that is repeated elsewhere in the genome can cause misalignment and thereby reduce sequencing performance. Performance can be enhanced by selecting an alignment algorithm or an algorithm parameter that minimizes misalignment. In this case, the value for the alignment selector can be a function of the sequence context, e.g., the presence or absence of a sequence of length that is repeated at least a given number of times in the genome (or in the portion of the genome being analyzed).
2. The tumor type being analyzed. For example, a specific tumor type can be characterized by an increased rate of deletions. Thus, performance can be enhanced by selecting an alignment algorithm or algorithm parameter that is more sensitive to indels. In this case, the value for the alignment selector can be a function of the tumor type, e.g., an identifier for the tumor type. In some instances, the value is the identity of the tumor type, e.g., a solid tumor or a hematologic malignancy (or premaligancy).
3. The gene, type of gene, or genetic locus being analyzed. Oncogenes, by way of example, are often characterized by substitutions or in-frame indels. Thus, performance can be enhanced by selecting an alignment algorithm or algorithm parameter that is particularly sensitive to these variants and specific against other types of variants. Tumor suppressors are often characterized by frame-shift indels. Thus, sequencing performance can be enhanced by selecting an alignment algorithm or algorithm parameter that is particularly sensitive to these variants. Thus, performance can be enhanced by selecting an alignment algorithm or algorithm parameter matched with the subject interval to be evaluated. In this case, the value for the alignment selector can be a function of the gene, type of gene, or genetic locus, e.g., an identifier for gene, gene type, or microsatellite locus. In some instances, for example, the value is the identity of the gene.
4. The site (e.g., nucleotide position) being analyzed. In this case, the value for the alignment selector can be a function of the site or the type of site, e.g., an identifier for the site or site type. In an embodiment the value is the identity of the site. For example, if the gene containing the site is highly homologous with another gene, normal/fast short read alignment algorithms (e.g., BWA) may have difficulty distinguishing between the two genes, potentially necessitating more intensive alignment methods (e.g., Smith-Waterman) or even assembly (ARACHNE). Similarly, if the gene sequence contains low-complexity regions (e.g., AAAAAA), more intensive alignment methods may be necessary.
5. The variant, or type of variant, associated with the subject interval being evaluated, e.g., a substitution, insertion, deletion, translocation, or other rearrangement. Thus, performance can be enhanced by selecting an alignment algorithm or algorithm parameter that is more sensitive to the specific variant type. In this case, the value for the alignment selector can be a function of the type of variant, e.g., an identifier for the type of variant. In some instances, the value is the identity of the type of variant, e.g., a substitution.
6. The type of sample, e.g., a sample described herein. Sample type/quality can affect error (spurious observation of non-reference sequence) rates. Thus, performance can be enhanced by selecting an alignment algorithm or algorithm parameter that accurately models the true error rate in the sample. In this case the value for the alignment selector can be a function of the type of sample, e.g., an identifier for the sample type. In some instances, e.g., the value is the identity of the sample type.

Generally, the accurate detection of indel mutations is an exercise in alignment, as the spurious indel rate on the sequencing platforms described herein is relatively low (thus, even a handful of observations of correctly aligned indels can be strong evidence of mutation). Accurate alignment in the presence of indels can be difficult however (especially as indel length increases). In addition to the general issues associated with alignment, e.g., of substitutions, the indel itself can cause problems with alignment. For instance, a deletion of 2 bp of a dinucleotide repeat cannot be readily definitively placed. Both sensitivity and specificity can be reduced by incorrect placement of shorter (<l5 bp) apparent indel-containing reads. Larger indels (e.g., getting closer in magnitude to the length of individual reads, e.g., reads of 36 bp) can cause failure to align the read at all, making detection of the indel impossible in the standard set of aligned reads.

Databases of cancer mutations can be used to address these problems and improve performance. To reduce false positive indel discovery (i.e., to improve specificity), regions around commonly expected indels can be examined for problematic alignments due to sequence context and addressed similarly to substitutions. To improve sensitivity of indel detection, several different approaches of using information on the indels expected in cancer can be used. For example, short-reads containing expected indels can be simulated and alignment attempted. The alignments can be studied and alignment parameters can be adjusted for problematic indel regions, for instance, by reducing gap open/extend penalties or by aligning partial reads (e.g. the first or second half of a read).

Alternatively, initial alignment can be attempted not just with the normal reference genome, but also with alternate versions of the genome containing each of the known or likely cancer indel mutations. In this approach, reads of indels that initially failed to align, or aligned incorrectly, are placed successfully on the alternate (mutated) version of the genome.

In this way, indel alignment (and thus mutation calling) can be optimized for the expected cancer genes/sites. As used herein, a sequence alignment algorithm embodies a computational method or approach used to identify the location within the genome from which a given read sequence (e.g., a short-read sequence from next-generation sequencing) most likely originated by assessing the similarity between the read sequence and a reference sequence. Any of a variety of algorithms can be applied to the sequence alignment problem. Some algorithms are relatively slow, but allow relatively high specificity. These include, e.g., dynamic programming-based algorithms. Dynamic programming is a method for solving complex problems by breaking them down into simpler steps. Other approaches are relatively more efficient, but are typically not as thorough. These include, e.g., heuristic algorithms and probabilistic methods designed for large-scale database search.

Alignment parameters are used in alignment algorithms to adjust the performance of an algorithm, e.g., to produce an optimal global or local alignment between a read sequence and a reference sequence. Alignment parameters can give weights for match, mismatch, and indels. For example, lower weights may allow alignments that include more mismatches and indels.

The sensitivity of alignment can be increased when an alignment algorithm is selected, or an alignment parameter is adjusted, based on tumor type, e.g., a tumor type that tends to have a particular mutation or mutation type.

The sensitivity of alignment can be increased when an alignment algorithm is selected, or an alignment parameter is adjusted, based on a particular gene or locus type (e.g., oncogene, tumor suppressor gene, microsatellite region). Mutations in different types of cancer-associated genes can have different impacts on cancer phenotype. For example, mutant oncogene alleles are typically dominant. Mutant tumor suppressor alleles are typically recessive, which means that in most cases both alleles of a tumor suppressor genes must be affected before an effect is manifested.

The sensitivity of alignment can be adjusted (e.g., increased) when an alignment algorithm is selected, or an alignment parameter is adjusted, based on mutation type (e.g., single nucleotide polymorphism, indel (insertion or deletion), inversion, translocation, tandem repeat).

The sensitivity of alignment can be adjusted (e.g., increased) when an alignment algorithm is selected, or an alignment parameter is adjusted, based on mutation site (e.g., a mutation hotspot). A mutation hotspot refers to a site in the genome where mutations occur up to 100 times more frequently than the normal mutation rate.

The sensitivity/specificity of alignment can be adjusted (e.g., increased) when an alignment algorithm is selected, or an alignment parameter is adjusted, based on sample type (e.g., cfDNA sample, ctDNA sample, FFPE sample, or CTC sample).

### Mutation Calling:

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequencing reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing of a large number of diverse genetic events at a large number of diverse genomic loci (e.g., genes, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads: examining the base calls and quality scores; calculating the probability of observed bases and quality scores given a potential genotype; and assigning genotypes (e.g., using Bayes' rule)): removing false positives (e.g., using depth thresholds to reject SNPs with read depth much lower or higher than expected: local realignment to remove false positives due to small indels); and performing linkage disequilibrium (LD)/imputation-based analysis to refine the calls.

Equations used to calculate the genotype likelihood associated with a specific genotype and position are described in. e.g., Li. H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in. e.g., Browning, B. L. and Yu, Z. Am. J. Hum. Genet. 2009, 85(6):847-61. Examples of low-coverage SNP calling methods are described in, e.g., Li, Y., el al., Annu. Rev. Genomics Hum. Gener. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation-detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011: 21(6):936-9; and Li. H., et al. (2009), Bioinformatics 25(16):2078-9.

Methods for generating indel calls and individual-level genotype likelihoods include, e.g., the Dindel algorithm (Albers, C.A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S.Q. and Durbin R. Genome Res. 201 11;21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 March 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

In some instances, the mutation calling methods described herein may comprise one or more of:
(i) assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is based on a unique (as opposed to the other assignments) first and/or second values;
(ii) the assignment of method of (i), wherein at least 10, 20, 30, 40, 50, 60, 70, 80, 90, 100, 200, 300, 400, or 500 of the assignments are made with first values which are a function of a probability of a variant being present in less than 5, 10, or 20%, e.g., of the cells in a tumor type:
(iii) assigning a nucleotide value (e.g., calling a mutation) for at least X nucleotide positions, each of which of which being associated with a variant having a unique (as opposed to the other X-1 assignments) probability of being present in a tumor of type, e.g., the tumor type of said sample, wherein, optionally, each of said of X assignments is based on a unique (as opposed to the other X-1 assignments) first and/or second value (wherein X=2, 3, 5, 10, 20, 40, 50, 60, 70, 80, 90, 100, 200, 300, 400, or 500);
(iv) assigning a nucleotide value (e.g., calling a mutation) at a first and a second nucleotide position, wherein the likelihood of a first variant at said first nucleotide position being present in a tumor of type (e.g., the tumor type of said sample) is at least 2, 5, 10, 20, 30, or 40 times greater than the likelihood of a second variant at said second nucleotide position being present, wherein, optionally, each assignment is based on a unique (as opposed to the other assignments) first and/or second value;
(v) assigning a nucleotide value to a plurality of nucleotide positions (e.g., calling mutations), wherein said plurality comprises an assignment for variants falling into one or more, e.g., at least 3, 4, 5, 6, 7, or all, of the following probability percentage ranges: less than or equal to 0.01; greater than 0.01 and less than or equal to 0.02; greater than 0.02 and less than or equal to 0.03; greater than 0.03 and less than or equal to 0.04; greater than 0.04 and less than or equal to 0.05; greater than 0.05 and less than or equal to 0.1; greater than 0.1 and less than or equal to 0.2: greater than 0.2 and less than or equal to 0.5; greater than 0.5 and less than or equal to 1.0; greater than 1.0 and less than or equal to 2.0; greater than 2.0 and less than or equal to 5.0; greater than 5.0 and less than or equal to 10.0: greater than 10.0 and less than or equal to 20.0: greater than 20.0 and less than or equal to 50.0; and greater than 50 and less than or equal to 100.0%, wherein, a probability range is the range of probabilities that a variant at a nucleotide position will be present in a tumor type (e.g., the tumor type of said sample) or the probability that a variant at a nucleotide position will be present in the recited percentage (%) of the cells in a sample, a library from the sample, or library catch from that library, for a preselected type (e.g., the tumor type of said sample), and wherein, optionally, each assignment is based on a unique first and/or second value (e.g., unique as opposed to the other assignments in a recited probability range or unique as opposed to the first and/or second values for one or more or all of the other listed probability ranges);
(vi) assigning a nucleotide value (e.g., calling a mutation) for at least 1, 2, 3, 5, 10, 20, 40, 50, 60, 70, 80, 90, 100, 200, 300, 400, 500, 600, 700, 800, 900, or 1,000 nucleotide positions each, independently, having a variant present in less than 50, 40, 25, 20, 15, 10, 5, 4, 3, 2, 1, 0.5, 0.4, 0.3, 0.2, or 0.1% of the DNA in said sample, wherein, optionally, each assignment is based on a unique (as opposed to the other assignments) first and/or second value;
(vii) assigning a nucleotide value (e.g., calling a mutation) at a first and a second nucleotide position, wherein the likelihood of a variant at the first position in the DNA of said sample is at least 2, 5, 10, 20, 30, or 40 times greater than the likelihood of a variant at said second nucleotide position in the DNA of said sample, wherein, optionally, each assignment is based on a unique (as opposed to the other assignments) first and/or second value;
(viii) assigning a nucleotide value (e.g., calling a mutation) in one or more or all of the following:
(1) at least 1, 2, 3, 4 or 5 nucleotide positions having a variant present in less than 1% of the cells in said sample, of the nucleic acids in a library from said sample, or the nucleic acid in a library catch from that library;
(2) at least 1, 2, 3, 4 or 5 nucleotide positions having a variant present in 1-2% of the cells in said sample, of the nucleic acid in a library from said sample, or the nucleic acid in a library catch from that library;
(3) at least 1, 2, 3, 4 or 5 nucleotide positions having a variant present in greater than 2% and less than or equal to 3% of the cells in said sample, of the nucleic acid in a library from said sample, or the nucleic acid in a library catch from that library
(4) at least 1, 2, 3, 4 or 5 nucleotide positions having a variant present in greater than 3% and less than or equal to 4% of the cells in said sample, of the nucleic acid in a library from said sample, or the nucleic acid in a library catch from that library;
(5) at least 1, 2, 3, 4 or 5 nucleotide positions having a variant present in greater than 4% and less than or equal to 5% of the cells in said sample, of the nucleic acid in a library from said sample, or the nucleic acid in a library catch from that library;
(6) at least 1, 2, 3, 4 or 5 nucleotide positions having a variant present in greater than 5% and less than or equal to 10% of the cells in said sample, of the nucleic acid in a library from said sample, or the nucleic acid in a library catch from that library;
(7) at least 1, 2, 3, 4 or 5 nucleotide positions having a variant present in greater than 10% and less than or equal to 20% of the cells in said sample, of the nucleic acid in a library from said sample, or the nucleic acid in a library catch from that library:
(8) at least 1, 2 3, 4 or 5 nucleotide positions having a variant present in greater than 20% and less than or equal to 40% of the cells in said sample, of the nucleic acid in a library from said sample, or the nucleic acid in a library catch from that library;
(9) at least 1, 2 3, 4 or 5 nucleotide positions having a variant present at greater than 40% and less than or equal to 50% of the cells in said sample, of the nucleic acid in a library from said sample, or the nucleic acid in a library catch from that library; or
(10) at least 1, 2 3, 4 or 5 nucleotide positions having a variant present in greater than 50% and less than or equal to 100% of the cells in said sample, of the nucleic acid in a library from said sample, or the nucleic acid in a library catch from that library:
wherein, optionally, each assignment is based on a unique first and/or second value (e.g., unique as opposed to the other assignments in the recited range (e.g., the range in (1) of less than 1%) or unique as opposed to a first and/or second values for a determination in one or more or all of the other listed ranges); or
(ix) assigning a nucleotide value (e.g., calling a mutation) at each of X nucleotide positions, each nucleotide position, independently, having a likelihood (of a variant being present in the DNA of said sample) that is unique as compared with the likelihood for a variant at the other X-1 nucleotide positions, wherein X is equal to or greater than 1, 2, 3, 5, 10, 20, 40, 50, 60, 70, 80, 90, 100, 200, 300, 400, 500, 600, 700, 800, 900, or 1,000, and wherein each assignment is based on a unique (as opposed to the other assignments) first and/or second value.

In some instances, a “threshold value” is used to evaluate reads, and select from the reads a value for a nucleotide position, e.g., calling a mutation at a specific position in a gene. In some instances, a threshold value for each of a number of subject intervals is customized or fine-tuned. The customization or tuning can be based on one or more of the factors described herein, e.g., the type of cancer in a sample, the gene in which the subject interval (subgenomic interval or expressed subgenomic interval) to be sequenced is located, or the variant to be sequenced. This provides for calling that is finely tuned to each of a number of subject intervals to be sequenced.

In some instances, the method is particularly effective when a relatively large number of diverse subgenomic intervals are analyzed.

Thus, in another embodiment, the method comprises the following mutation calling method:
(i) acquiring, for each of said X subject intervals, a threshold value, wherein each of said acquired X threshold values is unique as compared with the other X-1 threshold values, thereby providing X unique threshold values;
(ii) for each of said X subject intervals, comparing an observed value which is a function of the number of reads having a nucleotide value at a nucleotide position with its unique threshold value, thereby applying to each of said X subject intervals its unique threshold value; and
(iii) optionally, responsive to the result of said comparison, assigning a nucleotide value to a nucleotide position, wherein X is equal to or greater than 2.

In some instances, the method includes assigning a nucleotide value to at least 2, 3, 5, 10, 20, 40, 50, 60, 70, 80, 90, 100, 200, 300, 400, 500, 600, 700, 800, 900, or 1,000 nucleotide positions, each having, independently, a first value that is a function of a probability that is less than 0.5, 0.4, 0.25, 0.15, 0.10, 0.05, 0.04, 0.03, 0.02, or 0.01.

In some instances, the method includes assigning a nucleotide value to at each of at least X nucleotide positions, each independently having a first value that is unique as compared with the other X-1 first values, and wherein each of said X first values is a function of a probability that is less than 0.5, 0.4, 0.25, 0.15, 0.10, 0.05, 0.04, 0.03, 0.02, or 0.01, wherein X is equal to or greater than 1, 2, 3, 5, 10, 20, 40, 50, 60, 70, 80, 90, 100, 200, 300, 400, 500, 600, 700, 800, 900, or 1,000.

In some instances, a nucleotide position in at least 20, 40, 60, 80, 100, 120, 140, 160 or 180, 200, 300, 400, 500, 600, 700, 800, 900, 1,000, 2,000, 3,000, 4,000, or 5,000 genes or genomic loci (e.g., microsatellite loci) is assigned a nucleotide value. In some instances, unique first and/or second values are applied to subject intervals in each of at least 10%, 20%, 30%, 40%, or 50% of said genes or genomic loci analyzed.

In some instances, the method may comprise optimizing threshold values for a relatively large number of subject intervals, as is seen, e.g., in the following embodiments.

In some instances, a unique threshold value is applied to subject intervals, e.g., subgenomic intervals or expressed subgenomic intervals, in each of at least 3, 5, 10, 20, 40, 50, 60, 70, 80, 90, 100, 200, 300, 400, 500, 600, 700, 800, 900, 1,000, 2,000, 3,000, 4,000, or 5,000 different genes or genomic loci.

In some instances, a nucleotide position in at least 20, 40, 60, 80, 100, 120, 140, 160 or 180, 200, 300, 400, 500, 1,000, 2,000, 3,000, 4,000, or 5,000 genes or genomic loci is assigned a nucleotide value. In some instances, a unique threshold value is applied to a subject interval in each of at least 10%, 20%, 30%, 40%, or 50% of said genes or genomic loci analyzed.

In some instances, at least 10%, 20%, 30%, 40%, 50%, 60%, 70%. 80%, or 90% of the mutation calls made using the method are for subject intervals from genes or genomic loci described herein, e.g., microsatellite loci. In some instances, at least 10%, 20%, 30%, 40%, 50/o, 60%, 70%, 80%,o, or 90% of the unique threshold values described herein are for subject intervals from genes or genomic loci described herein, e.g., microsatellite loci. In some instances, at least 10%, 20%, 30%, 40%, 50%, 60%, 70%, 80%, or 90% of the mutation calls annotated, or reported, e.g., to a third party, are for subject intervals from genes or genomic loci, e.g., microsatellite loci.

In some instances, the assigned value for a nucleotide position is transmitted to a third party, optionally, with explanatory annotation. In some instances, the assigned value for a nucleotide position is not transmitted to a third party. In some instances, the assigned value for a plurality of nucleotide positions are transmitted to a third party, optionally, with explanatory annotations, and the assigned value for a second plurality of nucleotide positions are not transmitted to a third party.

In some instances, the method comprises assigning one or more reads to a subject, e.g., by barcode deconvolution.

In some instances, the method comprises assigning one or more reads as a tumor read or a control read, e.g., by barcode deconvolution. In some instances, the method comprises mapping, e.g., by alignment with a reference sequence, each of said one or more reads. In an embodiment, the method comprises memorializing a called mutation.

In some instances, the method comprises annotating a called mutation, e.g., annotating a called mutation with an indication of mutation structure, e.g., a missense mutation, or function, e.g., a disease phenotype. In some instances, the method comprises acquiring nucleotide sequence reads for tumor and control nucleic acid. In some instances, the method comprises calling a nucleotide value, e.g., a variant (e.g., a mutation), for each of the subject intervals (e.g., subgenomic intervals, expressed subgenomic intervals, or both), e.g., using a Bayesian calling method or a non-Bayesian calling method. In some instances, the method comprises evaluating a plurality of reads that include at least one SNP. In some instances, the method comprises determining a SNP allele ratio in the sample and/or control reads.

In some instances, the method further comprises building a database of sequencing/alignment artifacts for the targeted subgenomic regions. In some instances, the database can be used to filter out spurious mutation calls and improve specificity. In some instances, the database is built by sequencing unrelated samples or cell-lines, and recording non-reference allele events that appear more frequently than expected due to random sequencing error alone in one or more normal samples. This approach may classify germline variations as artifact, but that may be acceptable in a method concerned with somatic mutations. The misclassification of germline variation as artifact may be ameliorated if desired by filtering the database for known germline variations (removing common variants) and for artifacts that appear in only single individuals (removing rarer variations).

In some instances, the next-generation sequencing methods combined with alignment and/or mutation calling algorithms described herein can detect variant alleles present at an allele frequency of less than 20%, less than 10%, less than 5%, less than 4%, less than 3%, less than 2%, or less than 1% in a sample.

In some instances, the disclosed methods comprise assigning one or more reads to a subject, e.g., by barcode deconvolution. In some instances, the disclosed methods comprise assigning one or more reads as a tumor read or a control read, e.g., by barcode deconvolution.

In some instances, the method comprises mapping, e.g., by alignment with a reference sequence, each of said one or more reads. In some instances, the method comprises memorializing a called mutation. In some instances, the method comprises annotating a called mutation, e.g., annotating a called mutation with an indication of mutation structure, e.g., a missense mutation, or function, e.g., a disease phenotype.

In an embodiment, the method comprises acquiring nucleotide sequence reads for tumor and control nucleic acid. In some instances, the method comprises calling a nucleotide value, e.g., a variant, e.g., a mutation, for each of the subject intervals (e.g., sub genomic intervals, expressed subgenomic intervals, or both), e.g., using a Bayesian calling method or a non-Bayesian calling method.

In some instances, the method comprises evaluating a plurality of reads that include at least one SNP. In some instances, the method comprises determining an SNP allele ratio in the sample and/or control read.

In some instances, the method further comprises building a database of sequencing/alignment artifacts for the targeted subgenomic regions. In some instances, the database can be used to filter out spurious mutation calls and improve specificity. In some instances, the database is built by sequencing unrelated samples or cell-lines and recording non-reference allele events that appear more frequently than expected due to random sequencing error alone in one or more of these normal samples. This approach may classify germline variation as artifact, but that may be acceptable in a method concerned with somatic mutations. This misclassification of germline variation as artifact may be ameliorated if desired by filtering the database for known germline variations (e.g., removing common variants) and for artifacts that appear in only one individual (e.g., removing rare variants).

Additional description of mutation calling methods is provided in, e.g., International Patent Application Publication No. WO 2020/236941, the entire content of which is incorporated herein by reference.

### Systems:

Also disclosed herein are systems designed to implement the disclosed methods for determining MSI status in a sample from a subject. The systems may comprise, e.g., one or more processors, and a memory unit communicatively coupled to the one or more processors and configured to store instructions that, when executed by the one or more processors, cause the processor to: receive nucleic acid sequence data for a plurality of microsatellite loci in the sample; identify a set of microsatellite loci from the plurality of microsatellite loci based on a coverage requirement; apply a set of sequence-based exclusion criteria to the set of microsatellite loci to identify a subset of the set of microsatellite loci; calculate a microsatellite instability (MSI) score for the sample based on the number of microsatellite loci in the set and the number of microsatellite loci in the subset; compare the MSI score to a threshold (e.g., a first threshold); and if the MSI score is greater than or equal to the threshold, determine an MSI status of high microsatellite instability (MSI-H) for the sample, wherein the MSI status of high microsatellite instability may be indicative of a deficient DNA mismatch repair mechanism in a tissue of the subject. In some instances, the methods may further comprise comparing the MSI score to a second threshold if the MST score is less than the first (e.g., the predetermined) threshold; and if the MSI score is less than or equal to the second threshold, determining an MSI status of microsatellite stable (MSS) for the sample; if the MSI score is greater than the second threshold, determining an MSI status of equivocal microsatellite instability (MSI-E) for the sample.

In some instances, the disclosed systems may be used for determination of MSI status in any of a variety of samples as described herein (e.g., a tissue sample, biopsy sample, hematological sample, or liquid biopsy sample derived from the subject).

In some instances, the plurality of microsatellite loci for which sequencing data is processed to determine MSI status may comprise at least 20, 40, 60, 80, 100, 120, 140, 160 or 180, 200, 300, 400, 500, 600, 700, 800, 900, 1,000, 2,000, 3,000, 4,000, or 5,000 microsatellite loci.

In some instances, the microsatellite loci used for the determination of MSI status comprise mononucleotide, dinucleotide, trinucleotide repeat sequences (or motifs), or any combination thereof. In some instances, the microsatellite loci used for determination of MSI status comprise repeat sequences (or motifs) of 1, 2, 3, 4, 5, 6, 7, 8, 9, 10, 11, 12, 13, 14, 15, or more than 15 nucleotides (or base pairs) in length.

In some instances, the microsatellite loci used for determination of MSI status comprise 2, 3, 4, 5, 6, 7, 8, 9, 10, 15, 20, 25, 30, 35, 40, 45, 50, or more than 50 repeats of a given repeat sequence or motif.

In some instances, the microsatellite loci used for determination of MSI status comprise sequences (e.g., allele sequences) that have an overall length of at least 4 base pairs, at least 6, at least 8, at least 10, at least 12, at least 14, at least 16, at least 18, at least 20, at least 30, at least 40, at least 50, at least 60, at least 70, at least 80, at least 90, at least 100, at least 150, at least 200, at least 250, at least 300, at least 350, at least 400, at least 450, at least 500, at least 550, at least 600, at least 650, at least 700, or at least 750 base pairs.

In some instance, the nucleic acid sequence data is acquired using a next generation sequencing technique having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the exclusion criteria comprise removal of loci from the MSI score calculation that fail to meet a minimum sequencing coverage requirement of at least 75×, 100×, 150×, 150×, 200×, or 250×.

In some instances, the exclusion criteria comprise removal of loci that exhibit alleles that fail to meet a minimum allele frequency requirement (e.g., a minimum allele frequency requirement is at least 1%, 2%, 3%, 4%, 5%, 6%, 7%, 8%, 9%, or 10%), a removal of loci that exhibit alleles corresponding to known sequencing errors, a removal of loci that exhibit alleles corresponding to known germline alleles, or any combination thereof, from the calculation of MSI score. In some instances, the minimum allele frequency requirement is at least two standard deviations higher than a mean allele frequency as determined from a reference genome database. In some instances, the minimum allele frequency requirement is at least three standard deviations higher than a mean allele frequency as determined from a reference genome database. In some instances, the exclusion criteria are locus-dependent, that is, different criteria may be used for different microsatellite loci or subsets of microsatellite loci.

In some instances, the microsatellite instability (MSI) score is calculated as the number of microsatellite loci in the second subset divided by the number of microsatellite loci in the first subset.

In some instances, the first threshold is determined by iteratively comparing MSI scores for random subsets of a training data set to predictions obtained from a reference microsatellite instability assay. In some instances, the first threshold is set to a value that maximizes the sum of positive percent agreement (PPA) and negative percent agreement (NPA) with the predictions of the reference microsatellite instability assay, while simultaneously requiring that negative percent agreement be greater than 0.95. In some instances, the first threshold is set to a value of about 0.008, 0.009, about 0.010, about 0.011, about 0.012, about 0.013, or about 0.015.

In some instances, the second threshold is determined by iteratively comparing MSI scores for random subsets of a training data set to predictions obtained from a reference microsatellite instability assay. In some instances, the second threshold is set to a value that maximizes the sum of positive percent agreement (PPA) and negative percent agreement (NPA) with the predictions of the reference microsatellite instability assay, while simultaneously requiring that negative percent agreement be greater than 0.99. In some instances, the second threshold is set to a value of about 0.001, 0.002, about 0.003, about 0.004, or about 0.005.

In some instances, the microstatellite stability status for the sample is used to confirm a diagnosis of disease in the subject. In some instances, the microsatellite stability status for the sample is used to select, initiate, adjust, or terminate a treatment for cancer in the subject (e.g., a patient) from which the sample was derived, as described elsewhere herein. In some instances, the microsatellite stability status determined for a series of samples collected from the subject (e.g., a patient) at different points in time may be used to monitor disease progression, e.g., the progression of a cancer, as described elsewhere herein.

In some instances, the disclosed systems may further comprise a next generation sequencing platform (e.g., a Roche 454, Illumina Solexa, ABI-SOLiD, ION Torrent, or Pacific Bioscience sequencing platform), sample processing and library preparation workstations, microplate-handling robotics, fluid dispensing systems, temperature control modules, environmental control chambers, additional data storage modules, data communication modules (e.g., Bluetooth, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks:

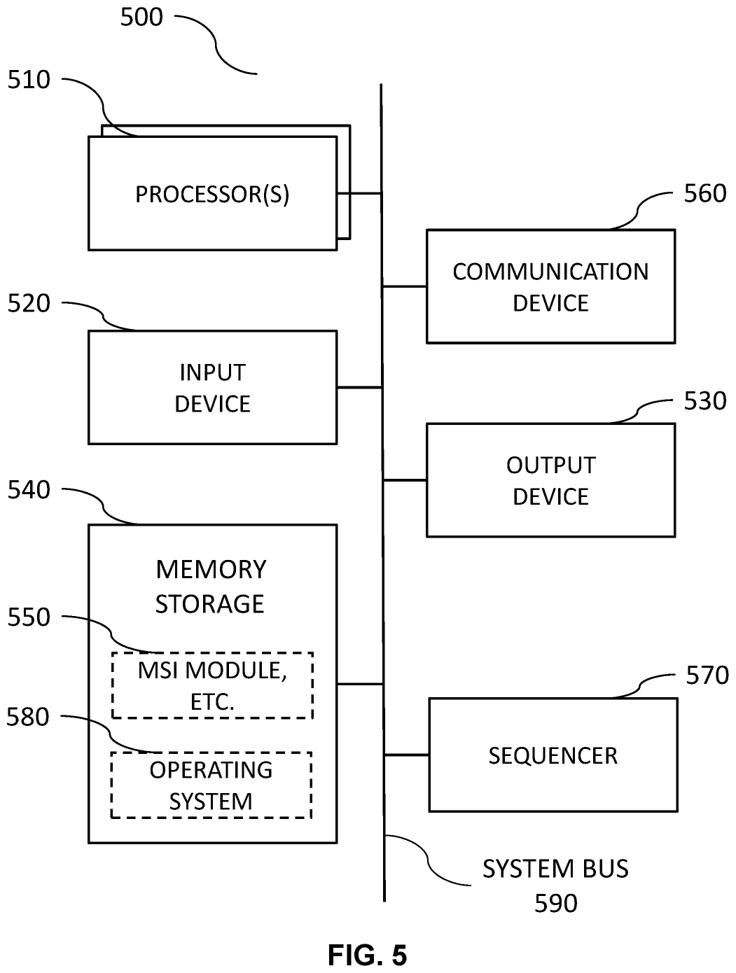

FIG. 5 illustrates an example of a computing device in accordance with one embodiment. Device 500 can be a host computer connected to a network. Device 500 can be a client computer or a server. As shown in FIG. 5, device 500 can be any suitable type of microprocessor-based device, such as a personal computer, workstation, server or handheld computing device (portable electronic device) such as a phone or tablet. The device can include, for example, one or more of processor(s) 510, input device 520, output device 530, storage 540, communication device 560, sequencer 570, operating system 580, and system bus 590. Input device 520 and output device 530 can generally correspond to those described herein, and can either be connectable or integrated with the computer.

Input device 520 can be any suitable device that provides input, such as a touch screen, keyboard or keypad, mouse, or voice-recognition device. Output device 530 can be any suitable device that provides output, such as a touch screen, haptics device, or speaker.

Storage 540 can be any suitable device that provides storage (e.g., an electrical, magnetic or optical memory including a RAM (volatile and non-volatile), cache, hard drive, or removable storage disk). Communication device 560 can include any suitable device capable of transmitting and receiving signals over a network, such as a network interface chip or device. The components of the computer can be connected in any suitable manner, such as via a wired media (e.g., a physical bus, ethernet, or any other wire transfer technology) or wirelessly (e.g., Bluetooth, Wi-Fi®, or any other wireless technology). For example, in FIG. 5, the components are connected by system bus 590.

Software module 550, which can be stored as executable instructions in storage 540 and executed by processor(s) 510, can include, for example, the MSI Status calling routine and/or other software modules that embody the functionality of the methods of the present disclosure (e.g., as embodied in the devices as described herein).

Software module 550 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 540, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 550 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

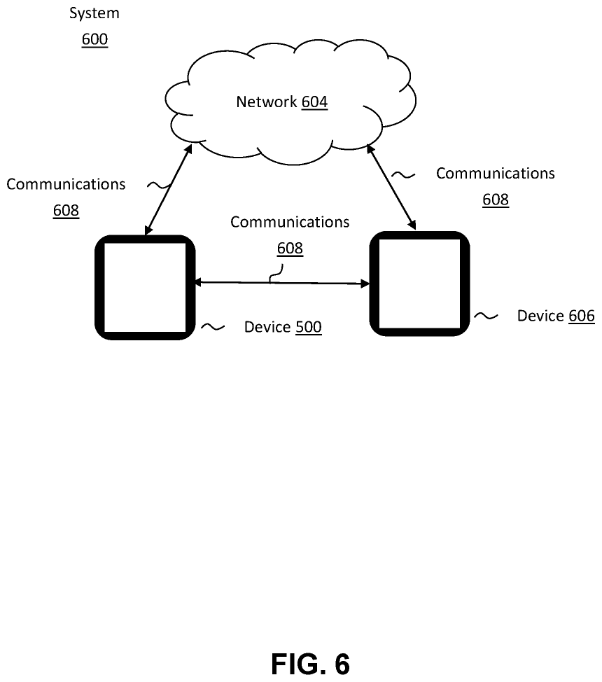

Device 500 may be connected to a network (e.g., network 604, as shown in FIG. 6 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 500 can implement any operating system (e.g., operating system 580) suitable for operating on the network. Software module 650 can be written in any suitable programming language, such as C. C++. Java or Python. In various embodiments, application software embodying the functionality of the present disclosure can be deployed in different configurations, such as in a client/server arrangement or through a Web browser as a Web-based application or Web service, for example. In some embodiments, operating system 580 is executed by one or more processors, e.g., processor(s) 510.

In some instances, device 500 can further include a sequencer 570, which can be any suitable sequencer, e.g., a next generation sequencer.

FIG. 6 illustrates an example of a computing system in accordance with one embodiment. In system 600, device 500 (e.g., as described above and illustrated in FIG. 5) is connected to network 604, which is also connected to device 606. In some embodiments, device 606 is a sequencer. Exemplary sequencers can include, without limitation, Roche/454's Genome Sequencer (GS) FLX System. Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

Devices 500 and 606 may communicate, e.g., using suitable communication interfaces via network 604, such as a Local Area Network (LAN), Virtual Private Network (VPN), or the Internet. In some embodiments, network 604 can be, for example, the Internet, an intranet, a virtual private network, a cloud network, a wired network, or a wireless network. Devices 500 and 606 may communicate, in part or in whole, via wireless or hardwired communications, such as Ethernet, IEEE 802.11b wireless, or the like. Additionally, devices 500 and 606 may communicate, e.g., using suitable communication interfaces, via a second network, such as a mobile/cellular network. Communication between devices 500 and 606 may further include or communicate with various servers such as a mail server, mobile server, media server, telephone server, and the like. In some embodiments, Devices 500 and 606 can communicate directly (instead of, or in addition to, communicating via network 604), e.g., via wireless or hardwired communications, such as Ethernet, IEEE 802.11b wireless, or the like. In some embodiments, devices 500 and 606 communicate via communications 608, which can be a direct connection or can occur via a network (e.g., network 604).

One or all of devices 500 and 606 generally include logic (e.g., http web server logic) or are programmed to format data, accessed from local or remote databases or other sources of data and content, for providing and/or receiving information via network 604 according to various examples described herein.

### EXAMPLES

### Example 1-Example of MSI Status Calculation

A non-limiting example of the calculation of an MSI score from the sequencing data for a sample is summarized in Tables 2-5.

TABLE 2
Loci Filtering

| | |
|---|---|
| Total number of loci initially: | 2838 |
| Loci removed due to minimum coverage threshold: | 85 |
| Number of evaluable loci: | 2753 |

TABLE 3
Allele Filtering

| | |
|---|---|
| Total number of alleles initially: | 5746 |
| Alleles removed due to minimum allele frequency | 2487 |
| threshold: | |
| Alleles removed as sequencing error or germline | 3257 |
| (germline database and reference genome match): | |
| Remaining Alleles: | 2 |

TABLE 4
MSI Score Calculation

| | |
|---|---|
| Loci with remaining alleles: | 2 |
| MSI Score: | 2/2753 = 0.0007 |

TABLE 5
MSI Score Thresholding

| | |
|---|---|
| Score ≥ 0.01237 | False |
| 0.0041 < Score < 0.01237 | False |
| Score ≤ 0.0041 | True |

The analysis begins by filtering out those microsatellite loci that fail to meet a minimum sequencing coverage threshold (Table 2), followed by filtering out alleles which fail to meet a minimum allele frequency threshold, or match known germline alleles or known sequencing artifacts (Table 3). The MSI score is calculated as the number of remaining microsatellite loci that have alleles that are not germline alleles, sequencing errors, and the like (two loci in this example), divided by the number of evaluable microsatellite loci (2,753 in this example), for a score of 0.0007 in this example (Table 4). The score is compared to a first threshold (e.g., a threshold of 0.01237 in this example) to determine if the sample should be assigned a status of microsatellite instability—high (MSI-H). If the score is less than the first threshold and greater than a second threshold (e.g., a threshold of 0.0041 in this example), the sample is assigned a status of microsatellite instability—equivocal (MSI-E). If the score is less than or equal to the second threshold, as is the case in this example, the sample is assigned a status of microsatellite stable (MSS) (Table 5).

### Example 2 Concordance of the Disclosed Methods for Determining MS status with a reference method

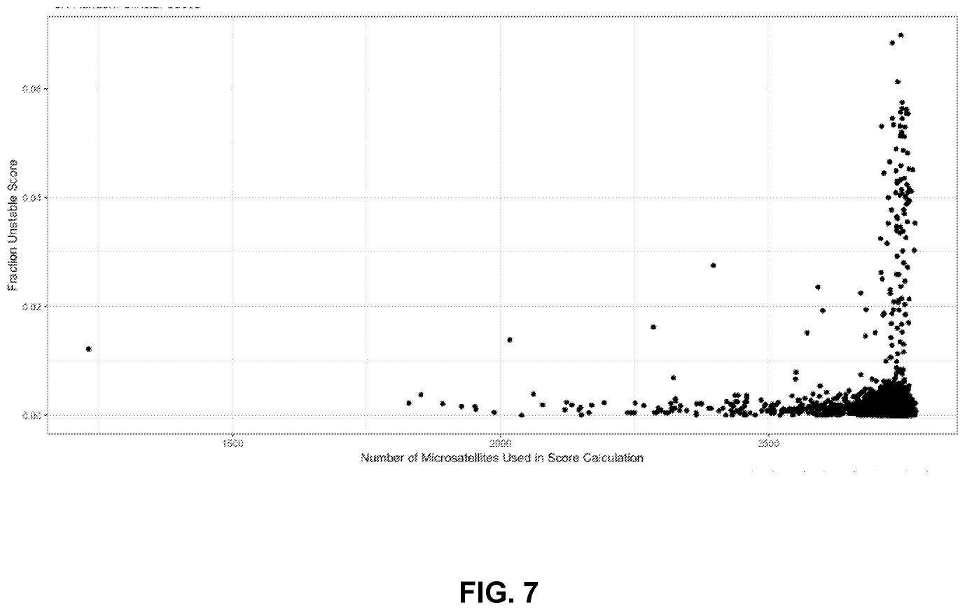

FIG. 7 provides a non-limiting example of data for the fraction of loci that were determined to be unstable (i.e., a “fraction unstable” or MSI score) in clinical samples versus the number of microsatellite loci evaluated and used to calculate the fraction unstable score. As can be seen, the fraction unstable score increased dramatically when larger numbers of microsatellite loci were used to calculate the fraction unstable score (or MSI score) according to the methods disclosed herein, thus indicating that accurate determination of the MSI status for a clinical sample required evaluation of a relatively large number of microsatellite loci.

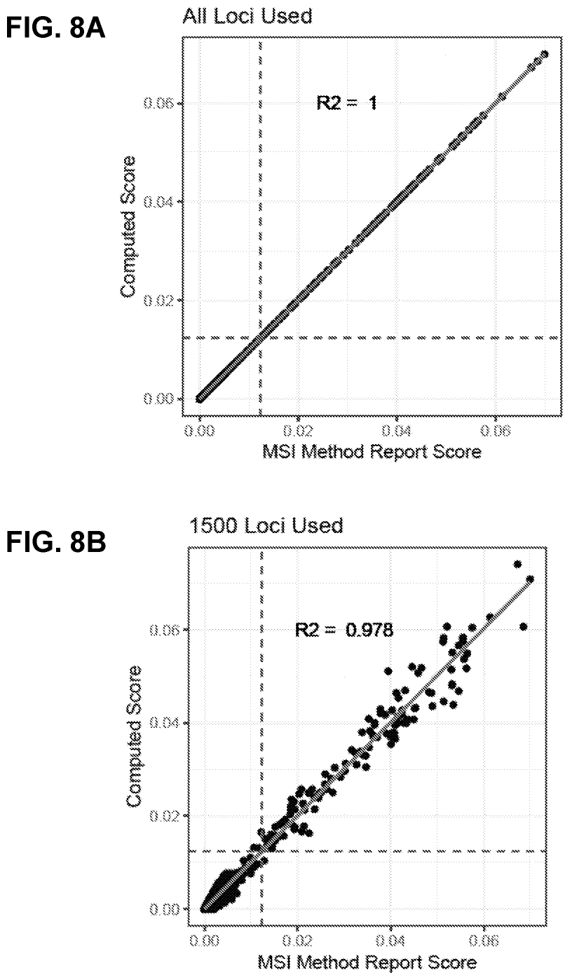

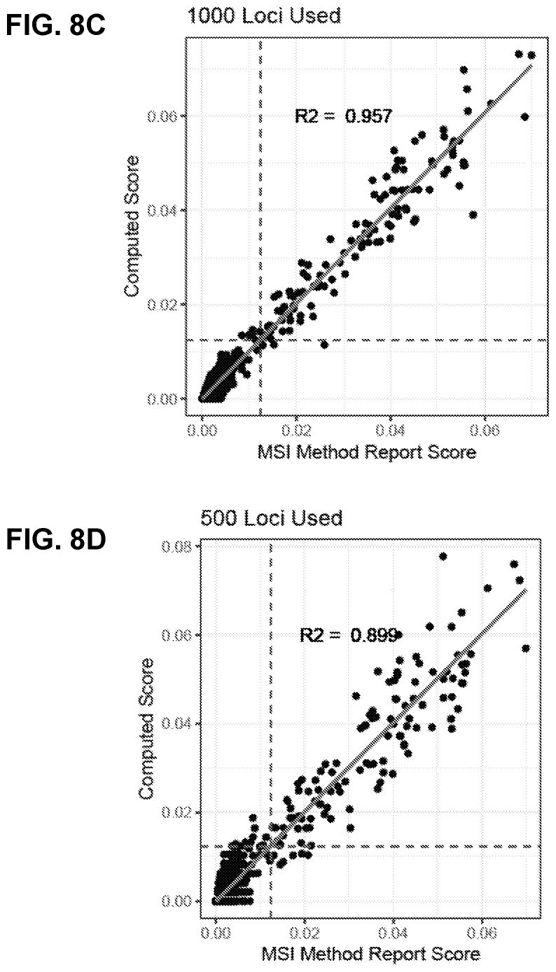

FIGS. 8A-D provide non-limiting examples of concordance data for MSI scores computed according to the disclosed methods with those obtained using a reference method (e.g., the Promega Microsatellite Instability (MSI) Assay from Promega Corporation, Madison, WI) as a function of different numbers of microsatellite loci used for the evaluation. FIG. 8A: all available/evaluable microsatellite loci used: a fit of the data to the expected linear relationship yielded a coefficient of determination (R²) value of 1.0. FIG. 8B: 1,500 microsatellite loci used; R²=0.978. FIG. 8C: 1,000 microsatellite loci used; R²=0.957. FIG. 8D: 500 microsatellite loci used; R²=0.899.

### Example 3—Limit—of Detection (LoD) & Accuracy for Determining MSI Status

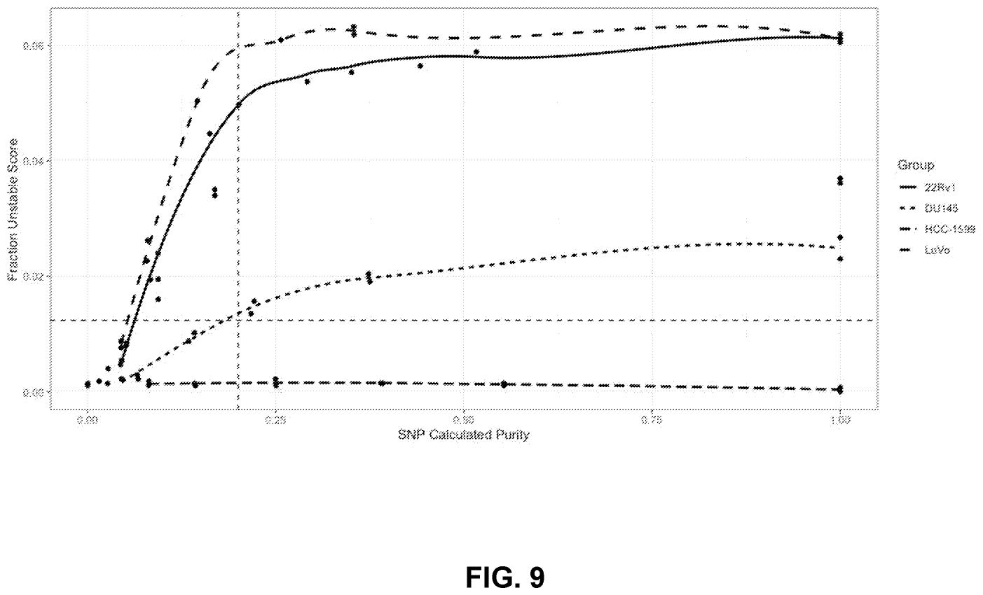

FIG. 9 provides a non-limiting example of data for the fraction unstable score plotted as a function of the amount of biomarker positive DNA from several known microsatellite unstable (MSI-H) samples mixed into normal DNA. The plotted curves were then used to determine a limit-of-detection (LOD) for detecting MSI-H status using the disclosed methods. The purity of the biomarker positive (microsatellite unstable) DNA was calculated based on the presence of a specified set of single nucleotide polymorphism (SNP) markers. Fraction unstable score data are plotted for DNA samples derived from the 22Rv1 (solid curve), DU145 (short dashed curve), HCC-1599 (lower long dashed curve), and LoVo (upper long dashed curve) cell lines that were diluted with NA12878 genomic DNA reference material. The horizontal dashed line indicates a cutoff threshold of 0.0124 used to determine a microsatellite instability status of MSI-H. The vertical dashed line indicates a target LoD of 20% microsatellite unstable DNA. The disclosed methods were able to detect microsatellite instability even at relative concentrations of less than 20% microsatellite unstable DNA.

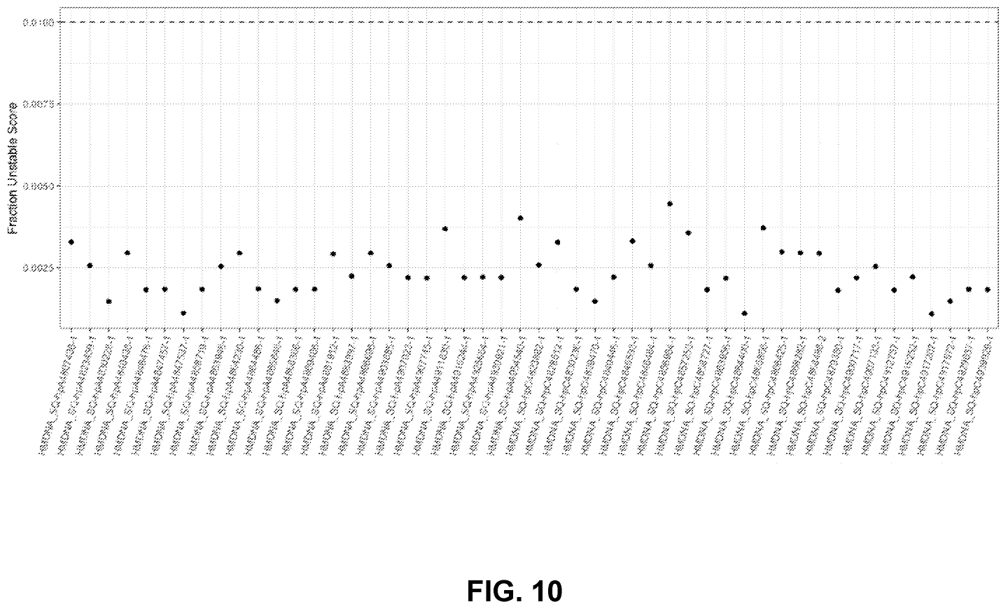

FIG. 10 provides a non-limiting example of data for fraction unstable score (MSI score) plotted for a series of negative control samples. Fraction unstable scores were determined for 50 randomly selected process control samples. There were approximately 2800 evaluable microsatellite loci in this study. Setting a 1% cutoff threshold (dashed horizontal line) for assigning a status of MSI-H corresponds to a finding that at least 28 unstable microsatellite loci are present in a given sample. On average, the process control samples had about 7 unstable microsatellite loci present.

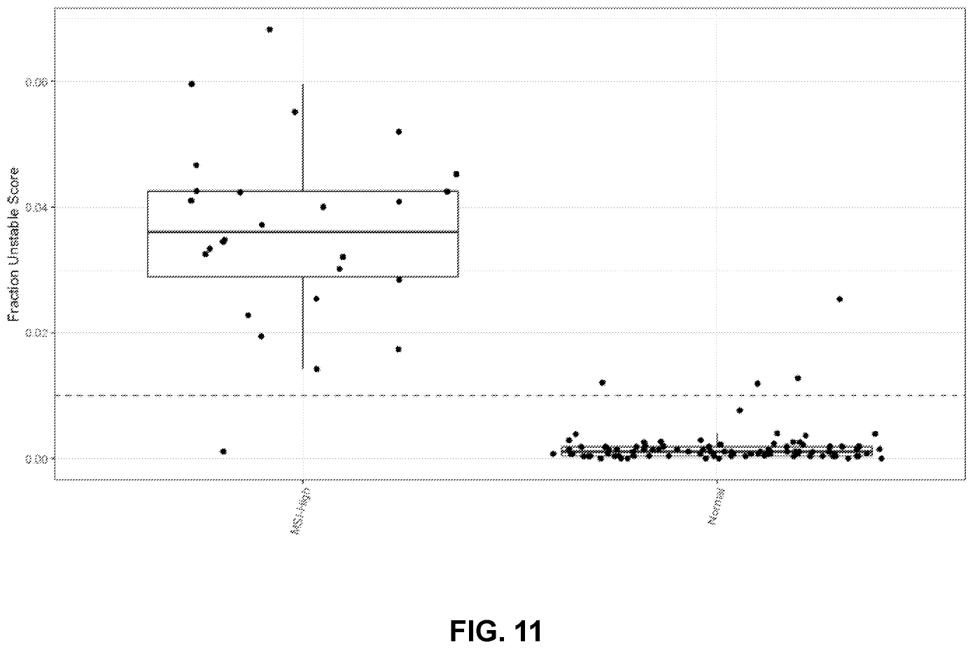

FIG. 11 provides a non-limiting example of data for fraction unstable scores plotted for microsatellite instability—high (MSI-H) samples and normal samples. The symbols indicate the median (horizontal line), interquartile range (IQR) (horizontal boxes), and the first quartile—1.5*IQR or the third quartile+1.5*IQR (vertical lines) for the fraction unstable scores in each category. The horizontal dashed line indicates the 1% cutoff threshold used for discriminating between normal and MSI-H status. The performance metrics for the assay indicated a true positive rate of 96%, a false positive rate of 4%, a true negative rate of 95%, a false negative rate of 4%, and an accuracy of 95%.

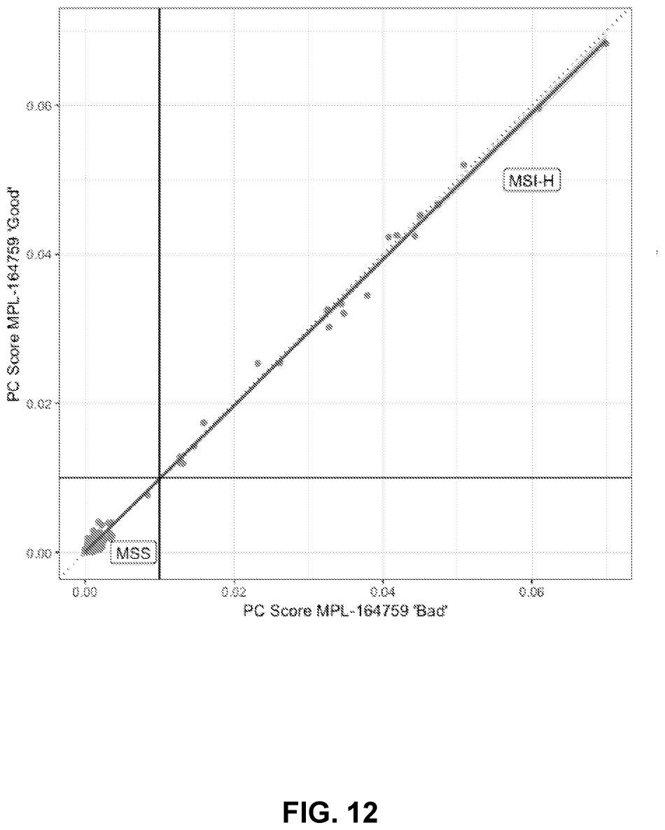

FIG. 12 provides a non-limiting example of data for MSI scores calculated for a series of samples that demonstrates the robustness of the disclosed methods to confounding effects of, for example, poor quality reagents used during the sample preparation and sequencing processes used to generate sequence data for selected microsatellite loci. Poor quality reagents led to higher false positive rates for detection of MSI-H status in samples when a less robust microsatellite instability algorithm was used for analysis.

### Example 4—Microsatellite Instability Versus Type of Cancer

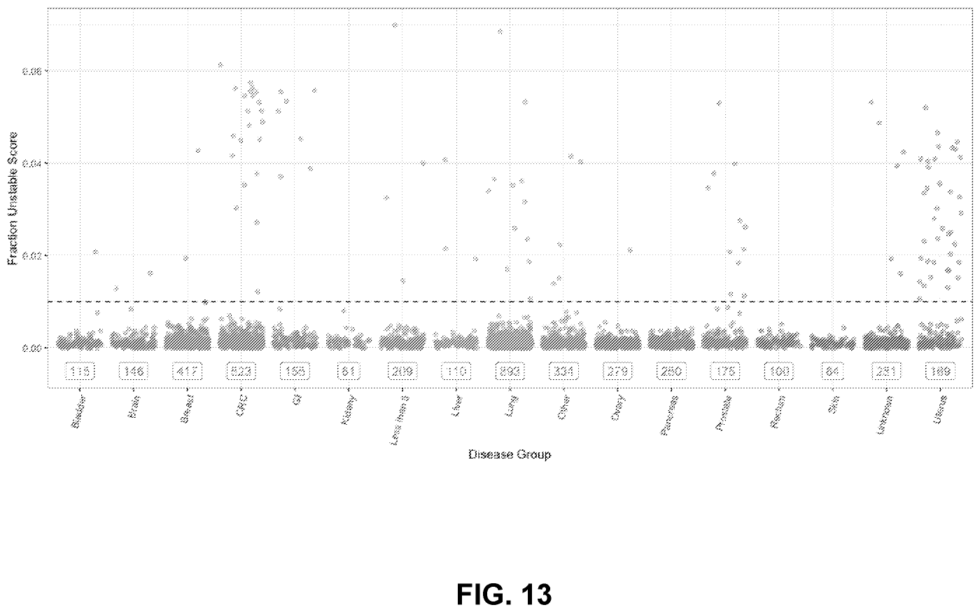

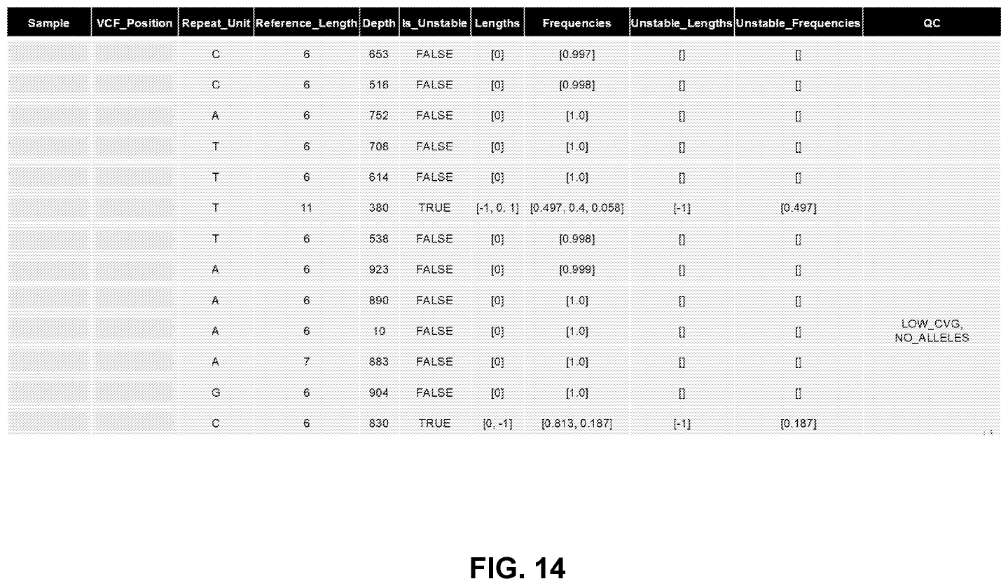

FIG. 13 provides a non-limiting example of fraction unstable scores for clinical samples grouped by disease type (the “less than 5” category comprised rare disease types for which the data was insufficient to separately examine MSI-H rates). Microsatellite instability scores were determined for 4,302 randomly selected clinical samples. Microsatellite instability was particularly prevalent for certain types of cancer, e.g., colorectal cancer (CRC), lung cancer, liver cancer, prostate cancer, and uterine cancer.

### Example 5—Exemplary Output File Formats

FIG. 14 provides a non-limiting example of the output provided for different microsatellite loci by a system for determining microsatellite instability according to the present disclosure. The output file may include, for example, sample identification information (column 1 from left), the microsatellite locus or position (column 2), the repeat unit for the locus (column 3), the length of the typical microsatellite allele at that locus as indicated in a reference database (column 4), the sequencing coverage depth at that locus (column 5), a determination of unstable status for the locus (column 6), the relative lengths (number of bases) of alleles identified at the locus compared to the reference allele length (column 7), the frequencies for the different alleles identified at the locus (column 8), the relative lengths of the unstable alleles (column 9), the frequencies for the unstable alleles (column 10), and any relevant sequencing quality control (QC) notes (column 11).

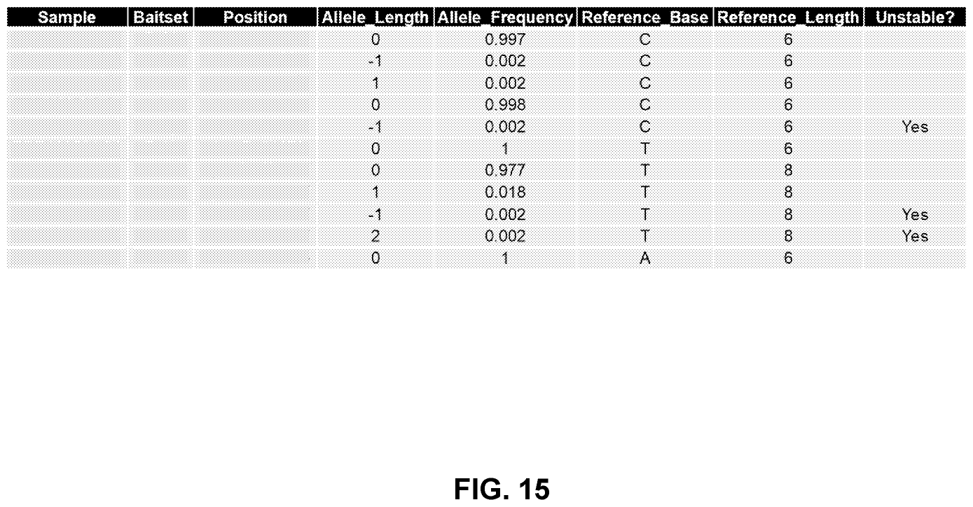

FIG. 15 provides a non-limiting example of the output provided for different alleles at a given microsatellite locus by a system for determining microsatellite instability according to the present disclosure. The output file may include, for example, sample identification information (column l from left), a name for the “baitset” or set of microsatellite loci targeted for analysis (column 2), the microsatellite locus or position for a given allele (column 3), the allele length relative to that in a reference database (column 4), the allele frequency (column 5), the repeated base in the reference database allele (column 6), the length of the allele (number of bases) in the reference database (column 7), and the determination of whether or not the allele represents an unstable allele (column 8).

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method for detecting a deficient DNA mismatch repair mechanism in a sample from a subject, the method comprising:
providing a plurality of nucleic acid molecules obtained from the sample;
ligating one or more adapters onto one or more nucleic acid molecules from the plurality of nucleic acid molecules;
amplifying nucleic acid molecules from the plurality of nucleic acid molecules;
capturing nucleic acid molecules from the amplified nucleic acid molecules;
sequencing, by a sequencer, the captured nucleic acid molecules to obtain a plurality of sequence reads corresponding to a plurality of microsatellite loci;
identifying, by one or more processors, a set of microsatellite loci from the plurality of microsatellite loci based on a coverage requirement;
applying, by the one or more processors, a set of sequence-based exclusion criteria to the set of microsatellite loci to identify a subset of the set of microsatellite loci;
determining, by the one or more processors, a microsatellite instability (MSI) score for the sample based on a number of microsatellite loci in the set and a number of microsatellite loci in the subset;
comparing, by the one or more processors, the MSI score to a threshold; and
determining an MSI status of high microsatellite instability for the sample if the MSI score is greater than or equal to the threshold, wherein the MSI status of high microsatellite instability is indicative of a deficient DNA mismatch repair mechanism in the sample.

2-22. (canceled)

23. The method of claim 1, wherein the sample is a tissue sample derived from the subject.

24. (canceled)

25. The method of claim 1, wherein the sample is a liquid biopsy sample comprising blood, plasma, cerebrospinal fluid, sputum, stool, urine, or saliva.

26. The method of claim 1, wherein the sample is a liquid biopsy sample and comprises circulating tumor cells (CTCs), cell-free DNA (cfDNA), circulating tumor DNA (ctDNA), or any combination thereof.

27-31. (canceled)

32. The method of claim 1, wherein each microsatellite locus in the plurality of microsatellite loci comprises an allele having a mononucleotide, dinucleotide, or trinucleotide repeat sequence at a minimum of 5× repeats, and having a total length of less than 50 base pairs.

33. (canceled)

34. The method of claim 1, wherein the coverage requirement is at least 75×, 100×, 150×, 150×, 200×, or 250×.

35. The method of claim 1, wherein the coverage requirement is locus-dependent.

36. The method of claim 1, wherein applying the set of sequence-based exclusion criteria comprises excluding, from the set of microsatellite loci, a microsatellite locus that comprises an allele having an allele frequency below an allele frequency requirement.

37. The method of claim 36, wherein the allele frequency requirement is at least 1%, at least 2%, at least 3%, at least 4%, at least 5%, at least 6%, at least 7%, at least 8%, at least 9%, or at least 10%.

38. The method of claim 1, wherein applying the set of sequence-based exclusion criteria comprises excluding, from the set of microsatellite loci, a microsatellite locus that comprises an erroneous allele sequence according to a statistical model.

39. The method of claim 1, wherein applying the set of sequence-based exclusion criteria comprises:
comparing a particular allele at a particular microsatellite locus from the set of microsatellite loci to a reference database of sequencing errors; and
excluding the particular microsatellite locus from the set of microsatellite loci if the particular allele corresponds to a known sequencing error.

40. The method of claim 39, wherein the particular microsatellite locus is excluded if the particular allele is an allele of less than 10 base pairs in length and the particular allele has an allele frequency less than or equal to a mean allele frequency plus two standard deviations for the particular allele in the reference database of sequencing errors.

41. The method of claim 39, wherein the particular microsatellite locus is excluded if the particular allele is an allele of greater than or equal to 10 base pairs in length and the particular allele has an allele frequency less than or equal to a mean allele frequency plus three standard deviations for the particular allele in the reference database of sequencing errors.

42. The method of claim 1, wherein applying the set of sequence-based exclusion criteria comprises comparing a particular allele at a particular microsatellite locus to one or more databases; and excluding the particular of microsatellite locus if the particular allele corresponds to a known germline allele.

43. The method of claim 1, wherein applying the set of sequence-based exclusion criteria comprises comparing a particular allele at a particular microsatellite locus to one or more databases; and excluding the particular microsatellite locus if the particular allele is equal in repeat length to a repeat length for the particular allele in the one or more databases, equal in overall length to an overall length for the particular allele in the reference human genome database, or equal in number of repeats to a number of repeats for the particular allele in the one or more databases.

44. The method of claim 1, wherein the set of sequence-based exclusion criteria is locus-dependent.

45. The method of claim 1, wherein the MSI score is calculated by comparing the number of microsatellite loci in the subset to the number of microsatellite loci in the set.

46. The method of claim 1, wherein the MSI status is used to diagnose or confirm a diagnosis of disease in the subject.

47. The method of claim 46, wherein the disease is cancer.

48. The method of claim 47, wherein the cancer is bladder cancer, brain cancer, breast cancer, colorectal cancer, gastrointestinal cancer, a hematological cancer, kidney cancer, liver cancer, lung cancer, osteogenic carcinoma, ovarian cancer, pancreatic cancer, prostate cancer, rectal cancer, skin cancer, and uterine cancer, leukemia, lymphomas, and endometrial cancer.

49. (canceled)

50. The method of claim 47, further comprising selecting a cancer therapy to administer to the subject based on the MSI score.

51. The method of claim 50, further comprising determining an effective amount of a cancer therapy to administer to the subject based on the MSI score.

52. (canceled)

53. The method of claim 50, wherein the cancer therapy comprises chemotherapy, radiation therapy, immunotherapy, surgery, or a therapy configured to target a defect in a DNA mismatch repair (MMR) pathway.

54-55. (canceled)

56. The method of claim 1, wherein the threshold is a first threshold, and the method further comprises:
comparing the MSI score to a second threshold if the MSI score is less than the first threshold;
calling an MSI status of microsatellite stable for the sample if the MSI score is less than or equal to the second threshold; and
calling an MSI status of equivocal microsatellite instability for the sample if the MSI score is greater than the second threshold.

57. The method of claim 1, wherein the threshold is a first threshold that is determined by:
performing a plurality of iterations to obtain a plurality of candidate first threshold values, and
averaging the plurality of candidate first threshold values to determine the first threshold.

58. The method of claim 57, wherein each iteration comprises:
randomly selecting a subset of a plurality of samples from a plurality of patients;
calculating a plurality of MSI scores for the subset; and
obtaining a candidate first threshold value that maximizes concordance with determinations of high microsatellite instability obtained using a reference microsatellite instability assay for the subset of the plurality of samples from the plurality of patients.

59. The method of claim 58, wherein the candidate first threshold value is set to a value that maximizes a sum of positive percent agreement (PPA) and negative percent agreement (NPA) with microsatellite instability status results obtained using the reference microsatellite instability assay, while requiring that NPA is greater than a first minimum requirement.

60-82. (canceled)

83. A system comprising:
one or more processors;
a memory storing one or more programs configured to be executed by the one or more processors, the one or more programs including instructions for:
receiving, by one or more processors, nucleic acid sequence data for a plurality of microsatellite loci in a sample;
identifying, by the one or more processors, a set of microsatellite loci from the plurality of microsatellite loci based on a coverage requirement;
applying, by the one or more processors, a set of sequence-based exclusion criteria to the set of microsatellite loci to identify a subset of the set of microsatellite loci;
determining, by the one or more processors, a microsatellite instability (MSI) score for the sample based on a number of microsatellite loci in the set and the number of microsatellite loci in the subset;
comparing, by the one or more processors, the MSI score to a threshold;
determining an MSI status of high microsatellite instability for the sample if the MSI score is greater than or equal to the threshold;
wherein the MSI status of high microsatellite instability is indicative of a deficient DNA mismatch repair mechanism in the sample.

84-111. (canceled)
